(19) United States
(12) Patent Application Publication
(10) Pub. No.: US 2010/0028951 A1
(43) Pub. Date: Feb. 4, 2010

(54) PRODUCTION OF GLYCOPROTEINS WITH MODIFIED FUCOSYLATION

(76) Inventor: Stephen Hamilton, Enfield, NH (US)

Correspondence Address: MERCK AND CO., INC, P O BOX 2000, RAHWAY, NJ, 07065-0907, US

(21) Appl. No.: 12/528,029

(22) PCT Filed: Mar. 3, 2008
(86) PCT No.: PCT/US08/02787

§ 371 Date: Aug. 20, 2009

Related U.S. Application Data
(60) Provisional application No. 60/905,345, filed on Mar. 7, 2007.

(51) Int. Cl. C12P 21/00; C12N 1/14; C12N 1/19; C12N 15/00

(52) U.S. Cl. 435/69.51; 435/254.11; 435/254.2; 435/254.23; 435/320.1; 435/69.1; 435/69.5; 435/69.6; 435/69.7

(57) ABSTRACT

Methods are disclosed for genetically engineering host cells that lack an endogenous pathway for fucosylating N-glycans of glycoproteins to be able to produce glycoproteins with fucosylated N-glycans.

### BACKGROUND OF THE INVENTION

(1) Field of the Invention

The present invention relates to the field of glycobiology, and in particular to methods for genetically engineering host cells that lack an endogenous pathway for fucosylating N-glycans of glycoproteins to be able to produce glycoproteins with fucosylated N-glycans.

(2) Description of Related Art

Therapeutic proteins intended for use in humans that are glycosylated should have complex, human N-glycosylation patterns. In general, it would be advantageous to produce therapeutic proteins using bacterial or eukaryotic microorganisms because of (a) the ability to rapidly produce high concentrations of protein; (b) the ability to use sterile, well-controlled production conditions (for example, GMP conditions); (c) the ability to use simple, chemically defined growth media; (d) ease of genetic manipulation; (e) the absence of contaminating human or animal pathogens; (f) the ability to express a wide variety of proteins, including those poorly expressed in cell culture owing to toxicity etc.; and, (g) ease of protein recovery (for example, via secretion into the medium). However, prokaryotes and lower eukaryotes do not normally produce proteins having complex N-glycosylation patterns. Therefore, animal cells are generally used to produce therapeutic proteins where it is desirable that the protein have a complex, human-like N-glycosylation pattern. But, there are a number of significant drawbacks to using animal cells for producing therapeutic proteins.

Only certain therapeutic proteins are suitable for expression in animal cells (for example, those lacking in any cytotoxic effect or other effect adverse to growth). Animal cell culture systems are usually very slow, frequently requiring over a week of growth under carefully controlled conditions to produce any useful quantity of the protein of interest. Protein yields nonetheless compare unfavorably with those from microbial fermentation processes. In addition, cell culture systems typically require complex and expensive nutrients and cofactors, such as bovine fetal serum. Furthermore, growth may be limited by programmed cell death (apoptosis).

Moreover, animal cells (particularly mammalian cells) are highly susceptible to viral infection or contamination. In some cases the virus or other infectious agent may compromise the growth of the culture, while in other cases the agent may be a human pathogen rendering the therapeutic protein product unfit for its intended use. Furthermore, many cell culture processes require the use of complex, temperature-sensitive, animal-derived growth media components, which may carry pathogens such as bovine spongiform encephalopathy (BSE) prions. Such pathogens are difficult to detect and/or difficult to remove or sterilize without compromising the growth medium. In any case, use of animal cells to produce therapeutic proteins necessitates costly quality controls to assure product safety.

Recently, it has been shown that lower eukaryotes, particularly yeast, can be genetically modified so that they express proteins having complex N-glycosylation patterns that are human-like or humanized. Such genetically modified lower eukaryotes can be achieved by eliminating selected endogenous glycosylation enzymes that are involved in producing high mannose N-glycans and introducing various combinations of exogenous enzymes involved in making complex N-glycans. Methods for genetically engineering yeast to produce complex N-glycans has been described in U.S. Pat. No. 7,029,872 and U.S. Published patent Application Nos. 2004/0018590, 2005/0170452, 2006/0286637, 2004/0230042, 2005/0208617, 2004/0171826, 2005/0208617, and 2006/0160179. For example, a host cell can be selected or engineered to be depleted in 1,6-mannosyl transferase activities, which would otherwise add mannose residues onto the N-glycan on a glycoprotein, and then further engineered to include each of the enzymes involved in producing complex, human-like N-glycans.

Animal and human cells have a fucosyltransferase pathway that adds a fucose residue to the GlcNAc residue at the reducing end the N-glycans on a protein. The fucosylation pathway in humans consists of a GDP-mannose dehydratase and GDP-keto-deoxy-mannose-epimerase/GDP-keto-deoxy-galactose-reductase (FX protein), both located in the cytoplasm, which in concert converts GDP-mannose to GDP-fucose; a GDP-fucose transporter located in the membrane of the Golgi apparatus, which transports the GDP-fucose into the Golgi apparatus; and a fucosyltransferase (Fut8), which transfers the fucose residue by means of an 1,6-linkage to the 6 position of the GlcNAc residue at the reducing end of the N-glycan. In contrast to higher eukaryotes, many lower eukaryotes, for example yeast, lack the enzymes involved in the fucosyltransferase pathway, produce glycoproteins that do not contain fucose (See for example, Bretthauer/Catellino, Biotechnol. Appl. Biochem. 30: 193-200 (1999); Rabina et al., Anal. Biochem. 286: 173-178 (2000)). However, the lack of fucose on glycoproteins has been shown to have advantages in certain cases. For example, in the production of monoclonal antibodies, immunoglobulin molecules, and related molecules, it has been shown that removal of the fucose sugar from the N-glycan of immunoglobulins increases or alters its binding to selected Ig receptors, which effects changes in properties such as antibody-dependent cellular cytotoxicity, or ADCC. (See, for example, U.S. Published Patent Application Nos. 2005/0276805 and US2003/0157108)

However, while removal of fucose from the N-glycans of immunoglobulins appears to enhance ADCC activity of the immunoglobulins, fucosylated N-glycans appear to be important for other glycoproteins. For example, the deletion of the fucosyltransferase gene in mice induces severe growth retardation, early death during post-natal development, and emphysema-like changes in the lung. These Fut8^{-/-} null mice were rescued from the emphysema-like phenotype by administration of exogenous TGF-beta1. Additionally, impaired receptor-mediated signaling was rescued by reintroduction of the Fut8 gene, showing that core fucosylation is crucial for proper functioning of growth factor receptors such as TGF-beta1 and EGF (Wang et al., Meth. Enzymol. 417: 11-22 (2006). In lung tissue derived from Fut8^{-/-} mice, the loss of core fucosylation impairs the function of low-density lipoprotein (LDL) receptor-related protein-1 (LRP-1), resulting in a reduction in the endocytosis of insulin like growth factor (IGF)-binding protein-3 (IGFBP-3) (Lee et al., J. Biochem. (Tokyo) 139: 391-8 (2006). In Fut8^{-/-} mouse embryonic fibroblast cells, α3β1 integrin-mediated cell migration is abolished and cell signaling is decreased, identifying the core fucose as essential for protein function (Zhao et al., J. Biol. Chem., 281: 38343-38350 (2006). In addition, there may be situations where it is desirable to produce antibody compositions where at least a portion of the antibodies in the compositions are fucosylated in order to decrease ADCC activity. Therefore, in particular cases it will be advantageous to provide lower eukaryotic organisms and cells capable of producing fucosylated glycopeptides. Accordingly, development of methods and materials for the production of lower eukaryotic host cells, such as fungi and yeast, and particularly yeasts such as Pichia pastoris, K lactis, and others, would facilitate development of genetically enhanced yeast strains for the recombinant production of fucosylated glycoproteins.

### BRIEF SUMMARY OF THE INVENTION

Accordingly, the present invention provides methods and materials for making lower eukaryotic expression systems that can be used to produce recombinant, fucosylated glycoproteins. In particular, provided are vectors containing genes encoding one or more of the enzymes involved in the mammalian fucosylation pathway and lower eukaryote host cells that have been transformed with the vectors to produce host cells that are capable of producing fucosylated glycoproteins. The vectors, host cells, and methods are particularly well adapted to use in expression systems based on yeast and fungal host cells, such as Pichia pastoris.

In one embodiment, the present invention provides methods and materials for transforming lower eukaryotic host cells with one or more vectors encoding the enzymatic activities for conversion of GDP-mannose into GDP-fucose and for attachment of fucose to an N-glycan produced by the host cell. In further embodiments, the present invention comprises hybrid vectors encoding a fusion protein comprising the catalytic domain of a fucosylation pathway enzyme fused to a non-native leader sequence, which encodes a targeting sequence that targets the fusion peptide to the appropriate location in the endoplasmic reticulum, the early Golgi apparatus, or the late Golgi apparatus. For example, the catalytic domain for the fucosyltransferase is fused to a leader peptide that targets the catalytic domain to a location within the endoplasmic reticulum, the early Golgi apparatus, or the late Golgi apparatus. In further embodiments, the lower eukaryote host cell is transformed with a vector encoding a GDP-fucose transferase which transports GDP-fucose from the cytoplasm to the interior of the Golgi.

The present invention provides a recombinant lower eukaryote host cell comprising a fucosylation pathway. In particular aspects, the host cell is yeast or filamentous fungus, for example, a yeast of the Pichia sp. such as Pichia pastoris.

In further aspects, the host cell further does not display α1,6-mannosyltransferase activity with respect to the N-glycan on a glycoprotein and includes an α1,2-mannosidase catalytic domain fused to a cellular targeting signal peptide not normally associated with the catalytic domain and selected to target α1,2-mannosidase activity to the ER or Golgi apparatus of the host cell whereby, upon passage of a recombinant glycoprotein through the ER or Golgi apparatus of the host cell, a recombinant glycoprotein comprising a fucosylated Man₅GlcNAc₂ glycoform is produced.

In further aspects, the above host cell further includes a GlcNAc transferase I catalytic domain fused to a cellular targeting signal peptide not normally associated with the catalytic domain and selected to target GlcNAc transferase I activity to the ER or Golgi apparatus of the host cell; whereby, upon passage of the recombinant glycoprotein through the ER or Golgi apparatus of the host cell, a recombinant glycoprotein comprising a fucosylated GlcNAcMan₅GlcNAc₂ glycoform is produced.

In further aspects, the above host cell further includes a mannosidase II catalytic domain fused to a cellular targeting signal peptide not normally associated with the catalytic domain and selected to target mannosidase II activity to the ER or Golgi apparatus of the host cell; whereby, upon passage of the recombinant glycoprotein through the ER or Golgi apparatus of the host cell, a recombinant glycoprotein comprising a fucosylated GlcNAcMan₃GlcNAc₂ glycoform is produced.

In further aspects, the above host cell further includes a GlcNAc transferase II catalytic domain fused to a cellular targeting signal peptide not normally associated with the catalytic domain and selected to target GlcNAc transferase II activity to the ER or Golgi apparatus of the host cell; whereby, upon passage of the recombinant glycoprotein through the ER or Golgi apparatus of the host cell, a recombinant glycoprotein comprising a fucosylated GlcNAc₂Man₃GlcNAc₂ glycoform is produced.

In further aspects, the above host cell further includes a Galactose transferase II catalytic domain fused to a cellular targeting signal peptide not normally associated with the catalytic domain and selected to target Galactose transferase II activity to the ER or Golgi apparatus of the host cell; whereby, upon passage of the recombinant glycoprotein through the ER or Golgi apparatus of the host cell, a recombinant glycoprotein comprising a fucosylated Gal₂GlcNAc₂Man₃GlcNAc₂ glycoform is produced.

In further aspects, the above host cell further includes a sialyltransferase catalytic domain fused to a cellular targeting signal peptide not normally associated with the catalytic domain and selected to target sialyltransferase activity to the ER or Golgi apparatus of the host cell; whereby, upon passage of the recombinant glycoprotein through the ER or Golgi apparatus of the host cell, a recombinant glycoprotein comprising a fucosylated NANA₂Gal₂GlcNAc₂Man₃GlcNAc₂ glycoform is produced.

Transforming the above host cells with a nucleic acid encoding a particular glycoprotein, compositions of the glycoprotein can be produced that comprise a plurality of glycoforms, each glycoform comprising at least one N-glycan attached thereto, wherein the glycoprotein composition thereby comprises a plurality of N-glycans in which a predominant glycoform comprises a desired fucosylated N-glycan. Depending upon the specific glycoprotein desired, the methods of the present invention can be used to obtain glycoprotein compositions in which the predominant N-glycoform is present in an amount between 5 and 80 mole percent greater than the next most predominant N-glycoform; in further embodiments, the predominant N-glycoform may be present in an amount between 10 and 40 mole percent; 20 and 50 mole percent; 30 and 60 mole percent; 40 and 70 mole percent; 50 and 80 mole percent greater than the next most predominant N-glycoform. In other embodiments, the predominant N-glycoform is a desired fucosylated N-glycoform and is present in an amount of greater than 25 mole percent; greater than 35 mole percent; greater than 50 mole percent; greater than 60 mole percent; or greater than 75 mole percent of the total number of N-glycans.

Thus, are provided host cells for producing glycoprotein compositions comprising a plurality of glycoforms, each glycoform comprising at least one N-glycan attached thereto, wherein the glycoprotein composition thereby comprises a plurality of fucosylated N-glycans in which the predominant N-glycan is selected from the group consisting of Man₅GlcNAc₂, GlcNAcMan₅GlcNAc₂, Man₃GlcNAc₂, GlcNAcMan₃GlcNAc₂, GlcNAc₂Man₃GlcNAc₂, GalGlcNAc₂Man₃GlcNAc₂, Gal₂GlcNAc₂Man₃GlcNAc₂, NANAGal₂GlcNAc₂Man₃GlcNAc₂, and NANA₂Gal₂GlcNAc₂Man₃GlcNAc₂.

In further aspects, greater than 25 mole percent of the plurality of fucosylated N-glycans consists essentially of a fucosylated glycoform in which the glycoform is selected from the group consisting of Man₅GlcNAc₂, GlcNAcMan₅GlcNAc₂, Man₃GlcNAc₂, GlcNAcMan₃GlcNAc₂, GlcNAc₂Man₃GlcNAc₂, GalGlcNAc₂Man₃GlcNAc₂, Gal₂GlcNAc₂Man₃GlcNAc₂, NANAGal₂GlcNAc₂Man₃GlcNAc₂, and NANA₂Gal₂GlcNAc₂Man₃ GlcNAc₂.

In further still aspects, greater than 25 mole percent; greater than 35 mole percent; greater than 50 mole percent; greater than 60 mole percent; greater than 75 mole percent; or greater than 90 mole percent of the plurality of N-glycans consists essentially of a fucosylated glycoform in which the glycoform is selected from the group consisting of Man₅GlcNAc₂, GlcNAcMan₅GlcNAc₂, Man₃GlcNAc₂, GlcNAcMan₃GlcNAc₂, GlcNAc₂Man₃GlcNAc₂, GalGlcNAc₂Man₃GlcNAc₂, Gal₂GlcNAc₂Man₃GlcNAc₂, NANAGal₂GlcNAc₂Man₃GlcNAc₂, and NANA₂Gal₂GlcNAc₂Man₃GlcNAc₂.

In the above glycoprotein composition, the fucose is in an α1,3-linkage with the GlcNAc at the reducing end of the N-glycan, an α1,6-linkage with the GlcNAc at the reducing end of the N-glycan, an α1,2-linkage with the Gal at the non-reducing end of the N-glycan, an α1,3-linkage with the GlcNac at the non-reducing end of the N-glycan, or an α1,4-linkage with a GlcNAc at the non-reducing end of the N-glycan.

Therefore, in particular aspects of the above the glycoprotein compositions, the glycoform is in an α1,3-linkage or α1,6-linkage fucose to produce a glycoform selected from the group consisting of Man₅GlcNAc₂(Fuc), GlcNAcMan₅GlcNAc₂(Fuc), Man₃GlcNAc₂(Fuc), GlcNAcMan₃GlcNAc₂(Fuc), GlcNAc₂Man₃GlcNAc₂(Fuc), GalGlcNAc₂Man₃GlcNAc₂(Fuc), Gal₂GlcNAc₂Man₃GlcNAc₂(Fuc), NANAGal₂GlcNAc₂Man₃GlcNAc₂(Fuc), and NANA₂Gal₂GlcNAc₂Man₃GlcNAc₂(Fuc); in an α1,3-linkage or α1,4-linkage fucose to produce a glycoform selected from the group consisting of GlcNAc(Fuc)Man₅GlcNAc₂, GlcNAc(Fuc)Man₃ GlcNAc₂, GlcNAc₂(Fuc₁₋₂)Man₃ GlcNAc₂, GalGlcNAc₂(Fuc₁₋₂)Man₃ GlcNAc₂, Gal₂GlcNAc₂(Fuc₁₋₂)Man₃ GlcNAc₂, NANAGal₂GlcNAc₂(Fuc₁₋₂)Man₃GlcNAc₂, and NANA₂Gal₂GlcNAc₂(Fuc₁₋₂)Man₃GlcNAc₂; or in an α1,2-linkage fucose to produce a glycoform selected from the group consisting of Gal(Fuc)GlcNAc₂Man₃ GlcNAc₂, Gal₂(Fuc₁₋₂)GlcNAc₂Man₃ GlcNAc₂, NANAGal₂(Fuc₁₋₁)GlcNAc₂Man₃ GlcNAc₂, and NANA₂Gal₂(Fuc₁₋₂)GlcNAc₂Man₃ GlcNAc₂.

In other aspects, the glycoprotein composition of the present invention comprise compositions wherein the above N-glycoform is present at a level from about 5 to 80 mole percent; 10 to 40 mole percent; 20 to 50 mole percent; 30 to 60 mole percent; 40 to 70 mole percent; or 50 to 80 mole percent greater than the next most predominant N-glycoform.

### DEFINITIONS

As used herein, the terms “N-glycan” and “glycoform” are used interchangeably and refer to an N-linked oligosaccharide, for example, one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. The predominant sugars found on glycoproteins are glucose (Glc), galactose (Gal), mannose (Man), fucose (Fuc), N-acetylgalactosamine (GalNAc), N-acetylglucosamine (GlcNAc) and sialic acid (for example, N-acetyl-neuraminic acid (NANA)). The processing of the sugar groups occurs co-translationally in the lumen of the ER and continues in the Golgi apparatus for N-linked glycoproteins.

N-glycans have a common pentasaccharide core of Man₃GlcNAc₂. N-glycans differ with respect to the number of branches (antennae) comprising peripheral sugars (for example, GlcNAc, galactose, fucose, and sialic acid) that are added to the Man₃GlcNAc₂ core structure which is also referred to as the “trimannose core”, the “pentasaccharide core”, or the “paucimannose core”. N-glycans are classified according to their branched constituents (for example, high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose or N-acetylgalactosamine residues that are optionally modified with sialic acid or derivatives (for example, “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). As an example, when a N-glycan comprises a bisecting GlcNAc on the trimannose core, the structure can be represented as Man₃GlcNAc₂(GlcNAc) or Man₃GlcNAc₃. When an N-glycan comprises a core fucose attached to the trimannose core, the structure may be represented as Man₃GlcNAc₂(Fuc). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. The various N-glycans are also referred to as “glycoforms.”

Abbreviations used herein are of common usage in the art, see, for example, abbreviations of sugars, above. Other common abbreviations include “PNGase”, or “glycanase” or “glucosidase” which all refer to peptide N-glycosidase F (EC 3.2.2.18).

The term “expression control sequence” as used herein refers to polynucleotide sequences that are necessary to affect the expression of coding sequences to which they are operatively linked. Expression control sequences are sequences that control the transcription, post-transcriptional events, and translation of nucleic acid sequences. Expression control sequences include appropriate transcription initiation, termination, promoter, and enhancer sequences; efficient RNA processing signals such as splicing and polyadenylation signals; sequences that stabilize cytoplasmic mRNA; sequences that enhance translation efficiency (for example, ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is necessary for expression, and can also include additional components whose presence is advantageous, for example, leader sequences and fusion partner sequences.

The term “recombinant host cell” (“expression host cell”, “expression host system”, “expression system” or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

The term “eukaryotic” refers to a nucleated cell or organism, and includes insect cells, plant cells, mammalian cells, animal cells, and lower eukaryotic cells.

The term “lower eukaryotic cells” includes yeast, fungi, collar-flagellates, microsporidia, alveolates (for example, dinoflagellates), stramenopiles (for example, brown algae, protozoa), rhodophyta (for example, red algae), plants (for example, green algae, plant cells, moss) and other protists. Yeast and fungi include, but are not limited to: Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. Pichia sp., any Saccharomyces sp., Hansenula polymorpha, any Kluyveromyces sp., Candida albicans, any Aspergillus sp., Trichoderma reesei, Chrysosporium lucknowense, any Fusarium sp., and Neurospora crassa.

The term “peptide” as used herein refers to a short polypeptide, for example, one that is typically less than about 50 amino acids long and more typically less than about 30 amino acids long. The term as used herein encompasses analogs and mimetics that mimic structural and thus biological function.

As used herein, the term “predominantly” or variations such as “the predominant” or “which is predominant” will be understood to mean the glycan species that has the highest mole percent (%) of total N-glycans after the glycoprotein has been treated with PNGase and released glycans analyzed by mass spectroscopy, for example, MALDI-TOF MS. In other words, the phrase “predominantly” is defined as an individual entity, such as a specific glycoform, is present in greater mole percent than any other individual entity. For example, if a composition consists of species A in 40 mole percent, species B in 35 mole percent and species C in 25 mole percent, the composition comprises predominantly species A.

Unless otherwise defined, all technical and scientific terms used herein have the same meaning as commonly understood by one of ordinary skill in the art to which this invention pertains. Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

### BRIEF DESCRIPTION OF THE DRAWINGS

FIG. 1 illustrates the fucosylation pathway present in many higher eukaryotic cells.

FIG. 2 shows the glyco-engineering steps required to produce a recombinant yeast capable of producing fucosylated glycoproteins. Endogenous GDP-Mannose, present in the yeast cytoplasm, is converted to GDP-Fucose by GDP-mannose-dehydratase (GMD) and the bifunctional enzyme FX. Subsequently, the product is translocated into the Golgi apparatus by the GDP-Fucose transporter (GFTr) and fucose is transferred onto the acceptor glycan by α-1,6-fucosyltransferase (FUT8). Enzymes are indicated by blue text and metabolic intermediates by black text. GDP-kdMan (GDP-4-keto-6-deoxy-mannose) and GDP-kdGal (GDP-4-keto-6-deoxy-galactose) are intermediates in the conversion of GDP-mannose to GDP-fucose.

FIG. 3A shows the vectors used in engineering yeast strains to produce fucosylated glycoproteins. Represented is the expression vector pSH995 into which the fucose biosynthetic and transfer genes are introduced. Introduction of the genes required for biosynthesis and transfer of fucose into pSH995 produced the vector pSH1022.

FIG. 3B shows the vector pSH1022. Shown in (B) are the flanking regions of the TRP2 loci used to integrate the genes into the Pichia genome; the dominant selection marker NATr; the GAPDH-CYC expression cassette; and the pUC19 plasmid backbone.

FIG. 4A shows a MALDI-TOF scan of the N-glycans released from the rat EPO demonstrating that Pichia pastoris strain YSH661 (strain RDP974 transformed with vector pSH1022 containing the fucosylation pathway genes) produced rEPO comprising Gal₂GlcNAc₂Man₃GlcNAc₂(Fuc) and Gal₂GlcNAc₂Man₃GlcNAc₂ N-glycans. The Gal₂GlcNAc₂Man₃GlcNAc₂(Fuc) N-glycans are within the box.

FIG. 4B shows a MALDI-TOF scan of the N-glycans released from the rat EPO control strain YSH660 (strain RDP974 transformed with control vector pSH995) produced a fucosylated or fucose-free Gal₂GlcNAc₂Man₃GlcNAc₂ N-glycans only.

### DETAILED DESCRIPTION OF THE INVENTION

The present invention provides methods and materials for the genetically engineering host cells capable of producing glycoproteins proteins that have fucosylated N-glycans. While the methods and materials are exemplified in the yeast Pichia pastoris, which does not possess an endogenous fucosylation pathway, the methods and materials may also be used to genetically engineer other lower eukaryotes such as fungi, prokaryote, and those higher eukaryotes, which do not have an endogenous fucosylation pathways, for example, insect cells. In other embodiments, the methods and materials may used to genetically engineer higher eukaryote cells that have an endogenous fucosylation pathway but where it is desirable to increase the amount of fucosylation present in glycoproteins produced by such host cells.

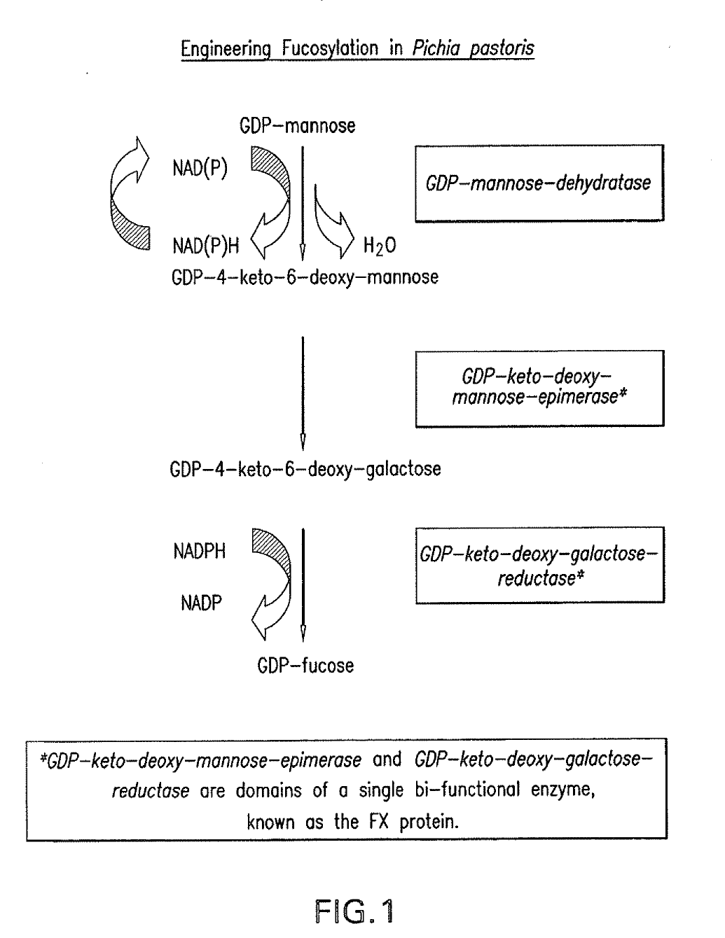

In general, the method of the present invention involves producing a host cell capable of producing fucosylated glycoproteins by introducing into the host cell nucleic acids encoding those enzymes or enzymatic activities involved in the fucosylation pathway that when introduced into the host cell will render the cell capable of producing fucosylated glycoproteins. These nucleic acids include, for example, nucleic acids encoding a GDP-mannose-4,6-dehydratase activity, a GDP-keto-deoxy-mannose-epimerase activity/GDP-keto-deoxy-galactose-reductase activity, a GDP-fucose transporter protein, and a fucosyltransferase activity. An overview of the fucosylation pathway in higher eukaryotes is shown in FIG. 1.

GDP-mannose-4,6-dehydratase (GMD) (EC 4.2.1.47) converts GDP-mannose to GDP-4-keto-6-deoxy-mannose in the presence of NAD has been identified in a number of species. The human GMP (hGMD) is encoded by the nucleotide sequence shown in SEQ ID NO: 1 and has the amino acid sequence shown in SEQ ID No: 2. Homologous genes with GDP-mannose-dehydratase activity include the porcine GMD, (Broschat et al., Eur. J. Biochem., 153(2):397-401 (1985)), Caenorhabditis elegans GMD and Drosophila melanogaster GMD (See, for example, Rhomberg et al., FEBS J.; 273:2244-56 (2006)); Arabidopsis thaliana; (See, e.g., Nakayama et al., Glycobiology; 13:673-80 (2003)); and E. coli, (Somoza et al., Structure, 8:123-35 (2000)).

GDP-keto-deoxy-mannose-epimerase/GDP-keto-deoxy-galactose-reductase (GDP-L-fucose synthase, EC 1.1.1.271) is a bifunctional enzyme, which has been identified in both eukaryotes and prokaryotes. The human GDP-keto-deoxy-mannose-epimerase/GDP-keto-deoxy-galactose-reductase is called the FX protein (also known as hFX or GER). The nucleotide sequence encoding the hFX is shown in SEQ ID NO: 3. The hFX protein has the amino acid sequence shown in SEQ ID NO: 4.

The GDP-fucose transporter has been identified in several species. The human GDP-fucose transporter (hGFTr) has been identified as related to congenital disorders of glycosylation-II (CDG-II) (Lubke et al., Nat. Genet. 28: 73-6 (2001)). Also known as Leukocyte Adhesion Deficiency II (LAD II), it appears that the disorder results from a disturbance in fucosylation of selectin ligands. Roos and Law, Blood Cells Mol. Dis. 27: 1000-4 (2001). The nucleotide sequence encoding the hGFTr is shown in SEQ ID NO: 5 and the amino acid sequence of the hGFTr is shown in SEQ ID NO: 6. Homologous genes with GDP-fucose transporter activity have been identified in other species, such as Drosophila melanogaster (Ishikawa et al., Proc. Natl. Acad. Sci. USA. 102:18532-7 (2005)), rat liver (Puglielli and Hirschberg; J. Biol. Chem. 274:35596-60 (1999)), and a putative CHO homolog (Chen et al., Glycobiology; 15:259-69 (2005)).

A number of fucosyltransferases have been identified (See Breton et al., Glycobiol. 8: 87-94 (1997); Becker, Lowe, Glycobiol. 13: 41R-53R (2003); Ma et al., Glycobiol. 16: 158R-184R (2006)), for example, α1,2-fucosyltransferase (EC 2.4.1.69; encoded by FUT1 and FUT2), α1,3-fucosyltransferase (glycoprotein 3-α-L-fucosyltransferase, EC 2.4.1.214; encoded by FUT3-FUT7 and FUT9), α1,4-fucosyltransferase (EC 2.4.1.65; encoded by FUT3), and α1,6-fucosyltransferase (glycoprotein 6-α-L-fucosyltransferase, EC 2.4.1.68; encoded by FUT8). In general, α1,2-fucosyltransferase transfer fucose to the terminal galactose residue in an N-glycan by way of an α1,2 linkage. In general, the α1,3-fucosyltransferase and α1,4-fucosyltransferases transfer fucose to a GlcNAc residue at the non-reducing end of the N-glycan.

In general, α1,6-fucosyltransferases transfer fucose by way of an α1,6-linkage to the GlcNAc residue at the reducing end of N-glycans (asparagine-linked GlcNAc). Typically, α1,6-fucosyltransferase requires a terminal GlcNAc residue at the non-reducing end of at least one branch of the trimannose core to be able to add fucose to the GlcNAc at the reducing end. However, an α1,6-fucosyltransferase has been identified that requires a terminal galactoside residue at the non-reducing end to be able add fucose to the GlcNAc at the reducing end (Wilson et al., Biochm. Biophys. Res. Comm. 72: 909-916 (1976)) and Lin et al. (Glycobiol. 4: 895-901 (1994)) has shown that in Chinese hamster ovary cells deficient for GlcNAc transferase I, the α1,6-fucosyltansferase will fucosylate Man₄GlcNAc₂ and Man₅GlcNAc₂ N-glycans. Similarly, α1,3-fucosyltransferase transfers to the GlcNAc residue at the reducing end of N-glycans but by way of an α1,3-linkage, generally with a specificity for N-glycans with one unsubstituted non-reducing terminal GlcNAc residue. The N-glycan products of this enzyme are present in plants, insects, and some other invertebrates (for example, Schistosoma, Haemonchus, Lymnaea). However, U.S. Pat. No. 7,094,530 describes an α1,3-fucosyltransferase isolated from human monocytic cell line THP-1.

The human α1,6-fucosyltransferase (hFUT8) has been identified by Yamaguchi et al., (Cytogenet. Cell. Genet. 84: 58-6 (1999)). The nucleotide sequence encoding the human FUT8 is shown in SEQ ID NO:7. The amino acid sequence of the hFUT8 is shown in SEQ ID NO: 8. Homologous genes with FUT8 activity have been identified in other species, such as a rat FUT8 (rFUT8) having the amino acid sequence shown in SEQ ID NO:10 and encoded by the nucleotide sequence shown in SEQ ID NO: 9; a mouse Fut8 (mFUT8) having the amino acid sequence shown in SEQ ID NO:12 and encoded by the nucleotide sequence shown in SEQ ID NO:11, and a porcine FUT8 (pFUT8) having amino acid sequence shown in SEQ ID NO:14 and encoded by the nucleotide sequence shown in SEQ ID NO:13. FUT8 has also been identified in CHO cells (Yamane-Ohnuki et al., Biotechnol. Bioeng. 87: 614-622 (2004)), monkey kidney COS cells (Clarke and Watkins, Glycobiol. 9: 191-202 (1999)), and chicken cells (Coullin et al., Cytogenet. Genome Res. 7: 234-238 (2002)). Paschinger et al., Glycobiol. 15: 463-474 (2005) describes the cloning and characterization of fucosyltransferases from C. elegans and D. melanogaster. Ciona intestinalis, Drosophila pseudoobscura, Xenopus laevis, and Danio rerio putative α1,6-fucosyltransferases have been identified (GenBank accession numbers AJ515151, AJ830720, AJ514872, and AJ781407, respectively).

The aforementioned fucosylation pathway enzymes or activities are encoded by nucleic acids. The nucleic acids can be DNA or RNA, but typically the nucleic acids are DNA because it preferable that the nucleic acids encoding the fucosylation pathway enzymes or activities are stably integrated into the genome of the host cells. The nucleic acids encoding the fucosylation pathway enzymes or activities are each operably linked to regulatory sequences that allow expression of the fucosylation pathway enzymes or activities. Such regulatory sequences include a promoter and optionally an enhancer upstream of the nucleic acid encoding the fucosylation pathway enzyme or activity and a transcription termination site downstream of the fucosylation pathway enzyme or activity. The nucleic acid also typically further includes a 5′ untranslated region having a ribosome binding site and a 3′ untranslated region having a polyadenylation site. The nucleic acid is often a component of a vector such as a plasmid, which is replicable in cells in which the fucosylation pathway enzyme or activity is expressed. The vector can also contain a marker to allow selection of cells transformed with the vector. However, some cell types, in particular yeast, can be successfully transformed with a nucleic acid that lacks vector sequences.

In general, the host cells transformed with the nucleic acids encoding the one or more fucosylation pathway enzymes or activities further includes one or more nucleic acids encoding desired glycoproteins. Like for the fucosylation pathway enzymes, the nucleic acids encoding the glycoproteins are operably linked to regulatory sequences that allow expression of the glycoproteins. The nucleic acids encoding the glycoproteins can be amplified from cell lines known to express the glycoprotein using primers to conserved regions of the glycoprotein (See, for example, Marks et al., J. Mol. Biol.: 581-596 (1991)). Nucleic acids can also be synthesized de novo based on sequences in the scientific literature. Nucleic acids can also be synthesized by extension of overlapping oligonucleotides spanning a desired sequence (See, for example, Caldas et al., Protein Engineering, 13: 353-360 (2000)).

The type of fucosylated N-glycan structure produced by the host cell will depend on the glycosylation pathway in the host cell and the particular fucosyltransferase. For example, α1,2-fucosyltransferases in general add a fucose to the terminal galactose on an N-glycan. As such, a pathway that utilizes an α1,2-fucosyltransferase would preferably be introduced into a host cell that is capable of producing N-glycans having a Gal₂GlcNAc₂Man₃GlcNAc₂ glycoform. The N-glycans produced will have fucose in an α1,2 linkage to the terminal galactose residues. Both α1,3-fucosyltransferases and α1,4-fucosyltransferases add fucose to one or more GlcNAc residues at or near the non-reducing end by way of an α1,3 or α1,4 linkage, respectively, or for some α1,3-fucosyltransferases, by way of an α1,3 linkage to the core GlcNAc linked to the asparagine residue of the glycoprotein. As such, a pathway that utilizes an α1,3/4-fucosyltransferase would preferably be introduced into a host cell that is capable of producing N-glycans having at least a GlcNAcMan₅GlcNAc₂ glycoform. Finally, α1,6-fucosyltransferases in general transfer fucose by way of an α1,6 linkage to the core GlcNAc linked to the asparagine residue of the glycoprotein. In general, a pathway that utilizes an α1,6-fucosyltransferase would preferably be introduced into a host cell that is capable of producing N-glycans having at least a GlcNAcMan₅GlcNAc₂, Man₅GlcNAc₂, or Man₄GlcNAc₂ glycoform.

The glycoproteins that can be produced in accordance using the methods disclosed herein include any desired protein for therapeutic or diagnostic purposes, regardless of the origin of the nucleic acid sequence for producing the glycoprotein. For example, monoclonal antibodies in which the N-glycan is not fucosylated have increased ADCC activity; however, increased ADCC activity is undesirable for monoclonal antibodies intended to bind receptor ligands as a treatment for a disorder but not elicit ADCC activity. Monoclonal antibodies produced in the host cells disclosed herein comprising the fucosylation pathway will have fucosylated N-glycans and will be expected to have decreased ADCC activity. As another example, immunoadhesins (See, U.S. Pat. Nos. 5,428,130, 5,116,964, 5,514,582, and 5,455,165; Capon et al. Nature 337:525 (1989); Chamow and Ashkenazi, Trends Biotechnol. 14: 52-60 (1996); Ashkenazi and Chamow, Curr. Opin. Immunol. 9: 195-200 (1997)), which comprise the extracellular portion of a membrane-bound receptor fused to the Fc portion of an antibody produced in the host cells disclosed herein comprising the fucosylation pathway will have fucosylated N-glycans and will be expected to have decreased ADCC activity. Examples of glycoproteins that can be produced according to the methods herein to have fucosylated N-glycans include, but are not limited to, erythropoietin (EPO); cytokines such as interferon-α, interferon-β, interferon-γ, interferon-ω, and granulocyte-CSF; coagulation factors such as factor VIII, factor IX, and human protein C; monoclonal antibodies, soluble IgE receptor α-chain, IgG, IgM, IgG, urokinase, chymase, and urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, osteoprotegerin tissue, plasminogen activator, G-CSF, GM-CSF, and TNF-receptor.

In particular embodiments, one or more of the nucleic acids encode fusion proteins comprising the catalytic domain of a fucosylation pathway protein fused to a targeting peptide, which targets the fusion protein to a particular region within the cell. Typically, the targeting peptide will target the fusion protein to a location within the secretory pathway. The term “secretory pathway” thus refers to organelles and components within the cell where glycoproteins are modified in preparation for secretion. The secretory pathway includes the endoplasmic reticulum (ER), the Golgi apparatus, the trans-Golgi network, and the secretory vesicles. For example, suitable cellular targeting peptides may target the catalytic domain to the ER, the Golgi apparatus, the trans-Golgi network, or secretory vesicles. Targeting peptides which may be useful in the present invention include those described in U.S. Pat. No. 7,029,872. In one embodiment, the catalytic domain of the fucosyltransferase is fused to a targeting peptide that directs the fusion protein to the Golgi apparatus. The particular targeting peptide fused to the fucosyltransferase catalytic domain will depend on the host cell, the particular fucosyltransferase, and the glycoprotein being produced. Examples of targeting peptides that can be used for targeting the fucosyltransferase have been disclosed in, for example, U.S. Pat. No. 7,029,872 and U.S. Published Patent Application Nos. 2004/0018590, 2004/0230042, 2005/0208617, 2004/0171826, 2006/0286637, and 2007/0037248.

The nucleic acids encoding the enzymes or activities involved in the fucosylation pathway are ligated into vectors, which are capable of being used to transfect host cells. Typically, the vectors will include regulatory elements, which have been isolated from the same species of cell as the intended host cell, or which have been isolated from other species, but which are known to be functional when inserted into the intended host cell. Typically, these regulatory elements include 5′ regulatory sequences, such as promoters, as well as 3′ regulatory sequences, such as transcription terminator sequences. Vectors will typically also include at least one selectable marker element that allows for selection of host cells that have been successfully transfected with the vector. The vectors are transferred into the intended host cells, and the resulting cells are screened for the presence of the selectable marker, to identify those host cells which have been successfully transfected with the vector, and which will therefore also carry the vector encoding the fusion protein.

Lower eukaryotes such as yeast are often preferred for expression of glycoproteins because they can be economically cultured, give high yields of protein, and when appropriately modified are capable of producing glycoproteins with particular predominant N-glycan structures. Yeast, in particular, offers established genetics allowing for rapid transformations, tested protein localization strategies and facile gene knock-out techniques. Various yeasts, such as K. lactis, Pichia pastoris, Pichia methanolica, and Hansenula polymorpha are commonly used for cell culture and production of proteins because they are able to grow to high cell densities and secrete large quantities of recombinant protein at an industrial scale. Likewise, filamentous fungi, such as Aspergillus niger, Fusarium sp, Neurospora crassa and others can be used to produce glycoproteins at an industrial scale.

Lower eukaryotes, particularly yeast, can be genetically modified so that they express glycoproteins in which the glycosylation pattern is complex or human-like or humanized. Such genetically modified lower eukaryotes can be achieved by eliminating selected endogenous glycosylation enzymes that are involved in producing high mannose N-glycans and introducing various combinations of exogenous enzymes involved in making complex N-glycans. Methods for genetically engineering yeast to produce complex N-glycans has been described in U.S. Pat. No. 7,029,872 and U.S. Published patent Application Nos. 2004/0018590, 2005/0170452, 2006/0286637, 2004/0230042, 2005/0208617, 2004/0171826, 2005/0208617, and 2006/0160179. For example, a host cell is selected or engineered to be depleted in 1,6-mannosyl transferase activities, which would otherwise add mannose residues onto the N-glycan on a glycoprotein. For example, in yeast, the OCH1 gene encodes 1,6-mannosyl transferase activity. The host cells is then further engineered to include one or more of the enzymes involved in producing complex, human-like N-glycans.

In one embodiment, the host cell further includes an α1,2-mannosidase catalytic domain fused to a cellular targeting signal peptide not normally associated with the catalytic domain and selected to target the α1,2-mannosidase activity to the ER or Golgi apparatus of the host cell. Passage of a recombinant glycoprotein through the ER or Golgi apparatus of the host cell produces a recombinant glycoprotein comprising a fucosylated Man₅GlcNAc₂ glycoform, for example a Man₅GlcNAc₂(Fuc) glycoform. U.S. Pat. No. 7,029,872 and U.S. Published Patent Application Nos. 2004/0018590 and 2005/0170452 disclose lower eukaryote host cells capable of producing a glycoprotein comprising a Man₅GlcNAc₂ glycoform.

In a further embodiment, the immediately preceding host cell further includes a GlcNAc transferase I (GnTI) catalytic domain fused to a cellular targeting signal peptide not normally associated with the catalytic domain and selected to target GlcNAc transferase I activity to the ER or Golgi apparatus of the host cell. Passage of the recombinant glycoprotein through the ER or Golgi apparatus of the host cell produces a recombinant glycoprotein comprising a fucosylated GlcNAcMan₅GlcNAc₂ glycoform, for example a GlcNAcMan₅GlcNAc₂(Fuc) glycoform. U.S. Pat. No. 7,029,872 and U.S. Published Patent Application Nos. 2004/0018590 and 2005/0170452 disclose lower eukaryote host cells capable of producing a glycoprotein comprising a GlcNAcMan₅GlcNAc₂ glycoform. The glycoprotein produced in the above cells can be treated in vitro with a hexoaminidase to produce a recombinant glycoprotein comprising a fucosylated Man₅GlcNAc₂(Fuc) glycoform.

In a further still embodiment, the immediately preceding host cell further includes a mannosidase II catalytic domain fused to a cellular targeting signal peptide not normally associated with the catalytic domain and selected to target mannosidase II activity to the ER or Golgi apparatus of the host cell. Passage of the recombinant glycoprotein through the ER or Golgi apparatus of the host cell produces a recombinant glycoprotein comprising a fucosylated GlcNAcMan₃GlcNAc₂ glycoform, for example a GlcNAcMan₃GlcNAc₂(Fuc) glycoform. U.S. Published Patent Application No. 2004/0230042 discloses lower eukaryote host cells that express mannosidase II enzymes and are capable of producing glycoproteins having predominantly a GlcNAc₂Man₃GlcNAc₂ glycoform. The glycoprotein produced in the above cells can be treated in vitro with a hexoaminidase to produce a recombinant glycoprotein comprising a Man₃GlcNAc₂(Fuc) glycoform.

In a further still embodiment, the immediately preceding host cell further includes GlcNAc transferase II (GnTII) catalytic domain fused to a cellular targeting signal peptide not normally associated with the catalytic domain and selected to target GlcNAc transferase II activity to the ER or Golgi apparatus of the host cell. Passage of the recombinant glycoprotein through the ER or Golgi apparatus of the host cell produces a recombinant glycoprotein comprising a fucosylated GlcNAc₂Man₃GlcNAc₂ glycoform, for example a GlcNAc₂Man₃GlcNAc₂(Fuc) glycoform. U.S. Pat. No. 7,029,872 and U.S. Published Patent Application Nos. 2004/0018590 and 2005/0170452 disclose lower eukaryote host cells capable of producing a glycoprotein comprising a GlcNAc₂Man₃GlcNAc₂ glycoform. The glycoprotein produced in the above cells can be treated in vitro with a hexoaminidase to produce a recombinant glycoprotein comprising a Man₃GlcNAc₂(Fuc) glycoform.

In a further still embodiment, the immediately preceding host cell further includes a Galactose transferase II catalytic domain fused to a cellular targeting signal peptide not normally associated with the catalytic domain and selected to target Galactose transferase II activity to the ER or Golgi apparatus of the host cell. Passage of the recombinant glycoprotein through the ER or Golgi apparatus of the host cell produces a recombinant glycoprotein comprising a fucosylated Gal₂GlcNAc₂Man₃GlcNAc₂ glycoform, for example a Gal₂GlcNAc₂Man₃GlcNAc₂(Fuc) glycoform. U.S. Published Patent Application No. 2006/0040353 discloses lower eukaryote host cells capable of producing a glycoprotein comprising a Gal₂GlcNAc₂Man₃GlcNAc₂ glycoform. The glycoprotein produced in the above cells can be treated in vitro with a galactosidase to produce a recombinant glycoprotein comprising a fucosylated GlcNAc₂Man₃GlcNAc₂ glycoform, for example a GlcNAc₂Man₃GlcNAc₂(Fuc) glycoform.

In a further still embodiment, the immediately preceding host cell further includes a sialyltransferase catalytic domain fused to a cellular targeting signal peptide not normally associated with the catalytic domain and selected to target sialyltransferase activity to the ER or Golgi apparatus of the host cell. Passage of the recombinant glycoprotein through the ER or Golgi apparatus of the host cell produces a recombinant glycoprotein comprising a fucosylated NANA₂Gal₂GlcNAc₂Man₃GlcNAc₂ glycoform, for example, a NANA₂Gal₂GlcNAc₂Man₃GlcNAc₂(Fuc) glycoform. For lower eukaryote host cells such as yeast and filamentous fungi, it is preferred that the host cell further include a means for providing CMP-sialic acid for transfer to the N-glycan. U.S. Published Patent Application No. 2005/0260729 discloses a method for genetically engineering lower eukaryotes to have a CMP-sialic acid synthesis pathway and U.S. Published Patent Application No. 2006/0286637 discloses a method for genetically engineering lower eukaryotes to produce sialylated glycoproteins. The glycoprotein produced in the above cells can be treated in vitro with a neuraminidase to produce a recombinant glycoprotein comprising a fucosylated Gal₂GlcNAc₂Man₃GlcNAc₂ glycoform, for example, a Gal₂GlcNAc₂Man₃GlcNAc₂(Fuc) glycoform.

Any one of the preceding host cells can further include one or more GlcNAc transferase selected from the group consisting of GnTIII, GnTIV, GnTV, GnTVI, and GnTIX to produce glycoproteins having bisected and/or multiantennary N-glycan structures such as disclosed in U.S. Published Patent Application Nos. 2004/074458 and 2007/0037248. Various of the preceding host cells further include one or more sugar transporters such as UDP-GlcNAc transporters (for example, Kluyveromyces lactis and Mus musculus UDP-GlcNAc transporters), UDP-galactose transporters (for example, Drosophila melanogaster UDP-galactose transporter), and CMP-sialic acid transporter (for example, human sialic acid transporter). Because lower eukaryote host cells such as yeast and filamentous fungi lack the above transporters, it is preferable that lower eukaryote host cells such as yeast and filamentous fungi be genetically engineered to include the above transporters.

In further embodiments of the above host cells, the host cells are further genetically engineered to eliminate glycoproteins having α-mannosidase-resistant N-glycans by deleting or disrupting the β-mannosyltransferase gene (BMT2)(See, U.S. Published Patent Application No. 2006/0211085) and glycoproteins having phosphomannose residues by deleting or disrupting one or both of the phosphomannosyl transferase genes PNO1 and MNN4B (See for example, U.S. Published Patent Application Nos. 2006/0160179 and 2004/0014170). In further still embodiments of the above host cells, the host cells are further genetically modified to eliminate O-glycosylation of the glycoprotein by deleting or disrupting one or more of the Dol-P-Man:Protein (Ser/Thr) Mannosyl Transferase genes (PMTs) (See U.S. Pat. No. 5,714,377).

It has been shown that codon optimization of genes or transcription units coding for particular polypeptides leads to increased expression of the encoded polypeptide, that is increased translation of the mRNA encoding the polypeptide. Therefore, in the case of the host cells disclosed herein, increased expression of the encoded enzymes will produce more of the encoded enzymes, which can lead to increased production of N-glycans that are fucosylated. In the context of codon optimization, the term “expression” and its variants refer to translation of the mRNA encoding the polypeptide and not to transcription of the polynucleotide encoding the polypeptide. The term “gene” as used herein refers to both the genomic DNA or RNA encoding a polypeptide and to the cDNA encoding the polypeptide.

Codon optimization is a process that seeks to improve heterologous expression of a gene when that gene is moved into a foreign genetic environment that exhibits a different nucleotide codon usage from the gene's native genetic environment or improve ectopic expression of a gene in its native genetic environment when the gene naturally includes one or more nucleotide codons that are not usually used in genes native to the genetic environment that encode highly expressed genes. In other words, codon optimization involves replacing those nucleotide codons of a gene that are used at a relatively low frequency in a particular genetic environment or organism with nucleotide codons that are used in genes that are expressed at a higher frequency in the genetic environment or organism. In that way, the expression (translation) of the gene product (polypeptide) is increased. The assumption is that the nucleotide codons that appear with high frequency in highly expressed genes are more efficiently translated than nucleotide codons that appear at low frequency.

In general, methods for optimizing nucleotide codons for a particular gene depend on identifying the frequency of the nucleotide codons for each of the amino acids used in genes that are highly expressed in an organism and then replacing those nucleotide codons in a gene of interest that are used with low frequency in the highly expressed genes with nucleotide codons that are identified as being used in the highly expressed genes (See for example Lathe, Synthetic Oligonucleotide Probes Deduced from Amino Acid Sequence Data: Theoretical and Practical Considerations, J. Molec. Biol.: 183: 1-12 (1985); Nakamura et al., Nuc. Acid Res. 28: 292 (2000); Fuglsang, Protein Expression & Purification 31: 247-249 (2003)). There are numerous computer programs that will automatically analyze the nucleotide codons of a nucleic acid of an organism encoding a gene and suggest nucleotide codons to replace nucleotide codons, which occur with low frequency in the organism, with nucleotide codons that are found in genes that are highly expressed in the organism.

The following examples are intended to promote a further understanding of the present invention.

### Example 1

This Example shows the construction of a Pichia pastoris strain capable of producing glycoproteins that include fucose in the N-glycan structure of the glycoprotein.

Escherichia coli strains TOP10 or XL10-Gold are used for recombinant DNA work. PNGase-F, restriction and modification enzymes are obtained from New England BioLabs (Beverly, Mass.), and used as directed by the manufacturer. α-1,6-Fucosidase is obtained from Sigma-Aldrich (St. Louis, Mo.) and used as recommended by the manufacturer. Oligonucleotides are obtained from Integrated DNA Technologies (Coralville, Iowa). Metal chelating “HisBind” resin is obtained from Novagen (Madison, Wis.). 96-well lysate-clearing plates are from Promega (Madison, Wis.). Protein-binding 96-well plates are from Millipore (Bedford, Mass.). Salts and buffering agents are from Sigma-Aldrich (St. Louis, Mo.).

### Amplification of Fucosylation Pathway Genes.

An overview of the fucosylation pathway is shown in FIG. 1. The open reading frame (ORF) of hGMD is amplified from human liver cDNA (BD Biosciences, Palo Alto, Calif.) using Advantage 2 polymerase following the procedure recommended by the manufacturer. Briefly, the primers SH415 and SH413 (5′-GGCGG CCGCC ACCAT GGCAC ACGCA CCGGC ACGCT GC-3′ (SEQ ID NO:15) and 5′-TTAAT TAATC AGGCA TTGGG GTTTG TCCTC ATG-3′ (SEQ ID NO:16), respectively) are used to amplify a 1,139 bp product from human liver cDNA using the following conditions: 97° C. for 3 minutes; 35 cycles of 97° C. for 30 seconds, 50° C. for 30 seconds, 72° C. for 2 minutes; and 72° C. for 10 minutes. Subsequently the product is cloned into pCR2.1 (Invitrogen, Carlsbad, Calif.), sequenced, and the resultant construct designated pSH985.

Using the conditions outlined above, the primers SH414 and SH411 (5′-GGCGG CCGCC ACCAT GGGTG AACCC CAGGG ATCCA TG-3′ (SEQ ID NO:17) and 5′-TTAAT TAATC ACTTC CGGGC CTGCT CGTAG TTG-3′ (SEQ ID NO:18), respectively) are used to amplify a 986 bp fragment from human kidney cDNA (BD Biosciences, Palo Alto, Calif.), which corresponds to the ORF of the human FX gene. Subsequently, this fragment is cloned into pCR2.1, sequenced, and designated pSH988.

The ORF of the human GFTr is amplified from human spleen cDNA (BD Biosciences, Palo Alto, Calif.) using the conditions outlined above, and the primers RCD679 and RCD680 (5′-GCGGC CGCCA CCATG AATAG GGCCC CTCTG AAGCG G-3′ (SEQ ID NO:19) and 5′-TTAAT TAATC ACACC CCCAT GGCGC TCTTC TC-3′ (SEQ ID NO:20), respectively). The resultant 1,113 bp fragment is cloned into pCR2.1, sequenced, and designated pGLY2133.

A truncated form of the mouse FUT8 ORF, encoding amino acids 32 to 575 and lacking the nucleotides encoding the endogenous transmembrane domain, is amplified from mouse brain cDNA (BD Biosciences, Palo Alto, Calif.) using the conditions outlined above and the primers SH420 and SH421 (5′-GCGGC GCGCC GATAA TGACC ACCCT GATCA CTCCA G-3′ (SEQ ID NO:21) and 5′-CCTTA ATTAA CTATT TTTCA GCTTC AGGAT ATGTG GG-3′ (SEQ ID NO:22), respectively). The resultant 1,654 bp fragment is cloned into pCR2.1, sequenced, and designated pSH987.

### Generation of Fucosylation Genes in Yeast Expression Cassettes.

Open reading frames for GMD, FX, and GFTr are generated by digesting the above vectors with NotI and PacI restriction enzymes to produce DNA fragments with a NotI compatible 5′ end and a PacI compatible 3′ end. The FUT8 fragment is generated by digesting with AscI and PacI restriction enzymes to produce a DNA with an AscI compatible 5′ end and a PacI compatible 3′ end.

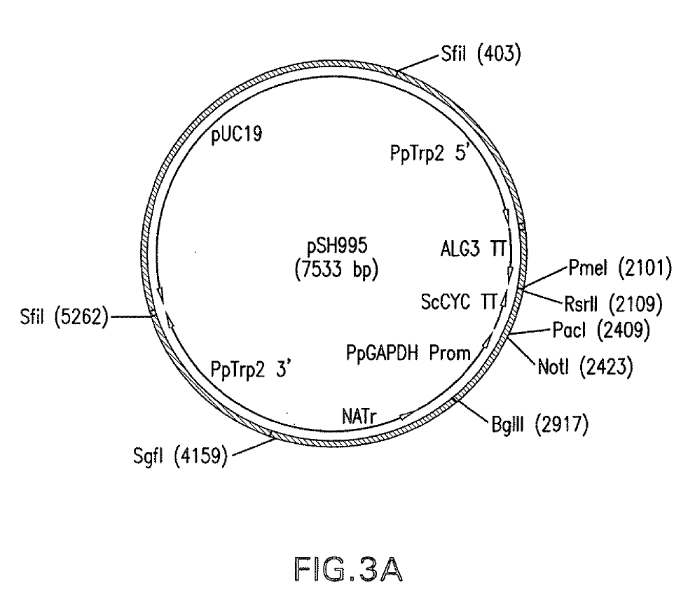

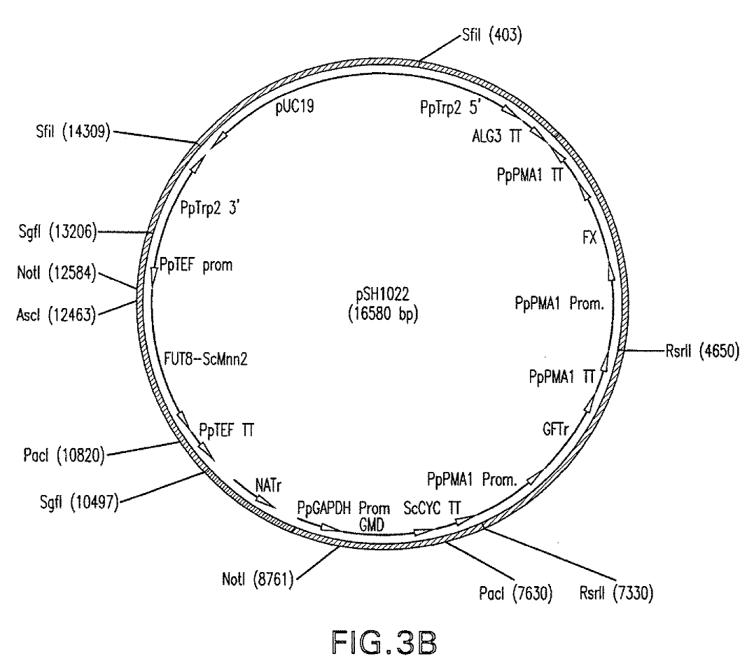

To generate the GMD expression cassette, GMD is cloned into yeast expression vector pSH995, which contains a P. pastoris GAPDH promoter and S. cerevisiae CYC transcription terminator sequence and is designed to integrate into the Pichia genome downstream of the Trp2 ORF, using the nourseothricin resistance marker. This vector is illustrated in FIG. 3A. The vector pSH985 is digested with NotI and PacI to excise a 1.1 Kb fragment containing the GMD ORF, which is then subcloned into pSH995 previously digested with the same enzymes. The resultant vector, containing GMD under the control of the GAPDH promoter, is designated pSH997. A.

To generate the FX expression cassette, the vector pSH988 is digested with NotI and PacI to excise a 1.0 Kb fragment containing the FX ORF, which is treated with T4 DNA polymerase to remove single strand overhangs (J. Sambrook, D. W. Russell, Molecular Cloning: A laboratory Manual (Cold Spring Harbor Laboratory Press, Cold Spring Harbor, N. Y., ed. 3rd, 2001)). Subsequently this fragment is subcloned into the vector pGLY359 (Hamilton et al., Science 313, 1441 (2006)) previously digested with NotI and AscI, and treated with T4 DNA polymerase. The resultant vector, pSH994, contains an FX expression cassette consisting of the FX ORF operably linked at the 5′ end to a P. pastoris PMA1 promoter (PpPMA1prom) and at the 3′ end to a P. pastoris PMA transcription terminator sequence (PpPMA1tt). The expression cassette is flanked by SwaI restriction sites.

The GFTr expression cassette is generated by digesting pGLY2133 with NotI and PacI to excise a 1.1 Kb fragment containing the GFTr ORF, which is treated with T4 DNA polymerase. Subsequently this fragment is subcloned into the vector pGLY363 (Hamilton, supra.), previously digested with NotI and PacI, and treated with T4 DNA polymerase. The resultant vector, pGLY2143, contains a GFTr expression cassette consisting of the GFTr ORF operably linked at the 5′ end to a PpPMA1prom and at the 3′ end to a PpPMA1tt. The expression cassette is flanked by RsrII restriction sites.

To generate the FUT8 catalytic domain fused to a yeast localization signal, the first 36 amino acids of S. cerevisiae targeting region of Mnn2 are analyzed by the GeneOptimizer software and codon-optimized for P. pastoris expression (GeneArt, Regensburg, Germany). The resultant synthetic DNA for ScMnn2 amino acids 1 to 36 is generated with 5′ NotI and 3′ AscI restriction enzyme compatible ends, cloned into a shuttle vector to produce plasmid vector pSH831. Subsequently, the vector pSH987 is digested with AscI and PacI to liberate a 1.6 Kb fragment encoding the FUT8 catalytic domain ORF, which is then subcloned in-frame to the DNA encoding the ScMnn2 targeting peptide in the vector pSH831, previously digested with the same enzymes. The resultant vector is designated pSH989. To generate the FUT8-ScMnn2 expression cassette, pSH989 is digested with NotI and PacI to release a 1.8 Kb fragment, which is subcloned into the vector pGLY361 (Hamilton et al., Science 313, 1441 (2006)) digested with the same enzymes. The resultant vector, pSH991, contains a FUT8-Mnn2 fusion protein consisting of the FUT8-Mnn2 fusion ORF operably linked at the 5′ end to a P. pastoris TEF promoter (PpTEFprom) and at the 3′ end to a P. pastoris TEF transcription terminator sequence (PpTEFtt). The expression cassette is flanked by SgrI restriction sites.

### Generation of Fucosylation Engineering Vector.

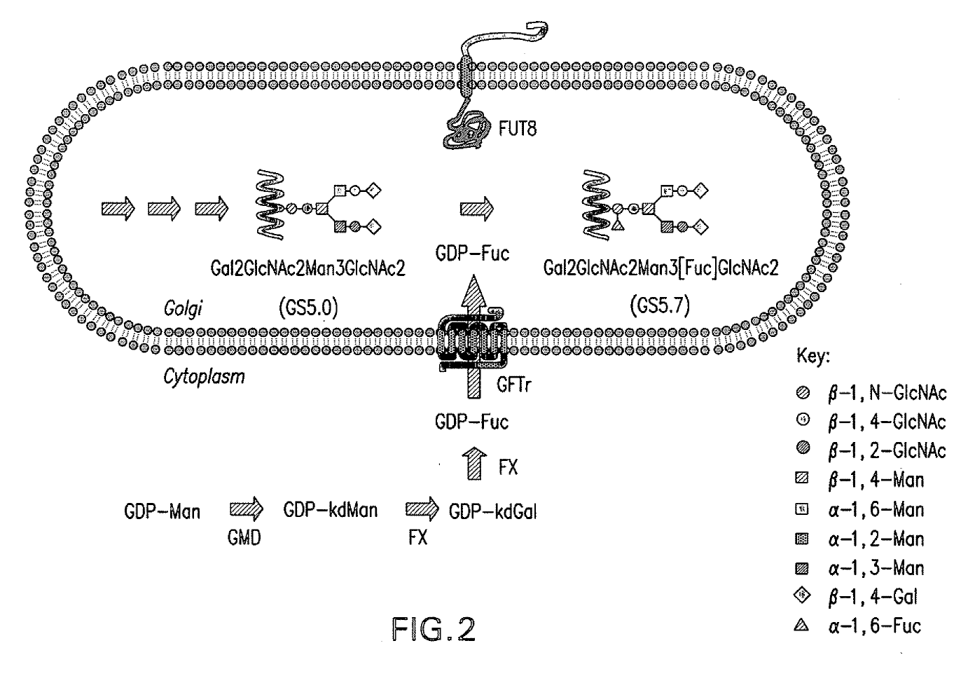

Vector pSH994 is digested with SwaI to release a 2.5 Kb fragment containing the FX expression cassette, which is subcloned into pSH997 (contains the GMD expression cassette) digested with PmeI. The resultant vector in which the PMA-FX and GAPDH-GMD expression cassettes are aligned in the same direction is designated pSH1009. The 2.7 Kb fragment containing the PMA-GFTr expression cassette is excised from pGLY2143 using the restriction enzyme RsrII and subcloned into pSH1009 digested with the same enzyme. The resultant vector in which the PMA-GFTr and GAPDH expression cassettes are aligned in the same direction is designated pSH1019. Finally, the 1.8 Kb TEF-FUT8 cassette is excised from pSH991 using SgfI and subcloned into pSH1019 digested with the same enzyme. The resultant vector in which the TEF-FUT8 and the GAPDH expression cassettes are aligned in the same direction is designated pSH1022. This vector is illustrated in FIG. 2B.

### Generation of Rat EPO Expression Vector.

A truncated form of Rattus norvegicus erythropoietin gene (rEPO), encoding amino acids 27 to 192, is amplified from rat kidney cDNA (BD Biosciences, Palo Alto, Calif.) using Advantage 2 polymerase as recommended by the manufacturer. Briefly, the primers-rEPO-forward and rEPO-reverse (5′-GGGAA TTCGC TCCCC CACGC CTCAT TTGCG AC-3′ (SEQ ID NO:23) and 5′-CCTCT AGATC ACCTG TCCCC TCTCC TGCAG GC-3′ (SEQ ID NO:24), respectively) are used to amplify a 516 bp product from rat kidney cDNA using the following cycling conditions: 1 cycle at 94° C. for 1 minute; 5 cycles at 94° C. for 30 seconds, 72° C. for 1 minute; 5 cycles at 94° C. for 30 seconds, 70° C. for 1 minute; 25 cycles at 94° C. for 20 seconds, 68° C. for 1 minute. Subsequently, the product is cloned into pCR2.1 (Invitrogen, Carlsbad, Calif.), sequenced, and the resultant construct designated pSH603. To generate the yeast expression vector, pSH603 is digested with EcoRI and XbaI to liberate a 506 bp fragment which was subcloned into pPICZαA (Invitrogen, Carlsbad, Calif.), which has previously been digested with the same enzymes. The resultant expression vector is designated pSH692. The rEPO in pSH692 is under the under the control of the AOX methanol-inducible promoter.

### Generation of Yeast Strains and Production of Rat EPO.

A P. pastoris glycoengineered cell line, YGLY1062, which is capable of producing recombinant glycoproteins having predominantly Gal₂GlcNAc₂Man₃GlcNAc₂ N-glycans (similar to the strains described in U.S. Published Patent Application No. 2006/0040353, which produce glycoproteins having Gal₂GlcNAc₂Man₃GlcNAc₂ N-glycans) is transformed with vector PSH692 to produce strain RDP974, which produces recombinant rat EPO (rEPO) with Gal₂GlcNAc₂Man₃GlcNAc₂ N-glycans. Strain RDP974 is similar to stain RDP762 described in Hamilton et al., Science 313, 1441-1443 (2006), which produces rat EPO having Gal₂GlcNAc₂Man₃GlcNAc₂ N-glycans.

The RPD974 strain has deletions in the OCH1, PNO1, MNN4B, and BMT2 genes and includes DNA encoding the full-length Kluyveromyces lactis UDP-GlcNAc transporter, M. musculus UDP-GlcNAc transporter, S. cerevisiae UDP-galactose 4-epimerase, and D. melanogaster UDP-Galactose transporter; and DNA encoding a M. musculus α1,2-Mannosidase I catalytic domain fused to DNA encoding amino acids 1-36 of an S. cerevisiae MNN2 leader sequence; DNA encoding the H. sapiens β1,2-GlcNAc transferase I (GnTI) catalytic domain fused to DNA encoding amino acids 1-36 of an S. cerevisiae MNN2 leader sequence; DNA encoding a Drosophila melanogaster Mannosidase II catalytic domain fused to DNA encoding amino acids 1-36 of an S. cerevisiae MNN2 leader sequence, DNA encoding a Rattus norvegicus β1,2-GlcNAc transferase II (GnTII) catalytic domain fused to DNA encoding amino acids 1-97 of an S. cerevisiae MNN2 leader sequence, and DNA encoding an H. sapiens β1,4-galactosyltransferase (GalTI) catalytic domain fused to DNA encoding amino acids 1-58 of an S. cerevisiae KRE2 (MNTI) leader sequence. U.S. Published Patent Application No. 2006/0040353 discloses methods for producing Pichia pastoris cell lines that produce galactosylated glycoproteins in lower yeast (See also, U.S. Pat. No. 7,029,872, U.S. Published Patent Application Nos. 2004/0018590, 2004/0230042, 2005/0208617, 2004/0171826, 2006/0286637, and 2007/0037248, and Hamilton et al., Science 313, 1441-1443 (2006).

Strain RDP974 is then used as the host strain for introducing the fucosylation pathway in vector pSH1022. Briefly, 10 μg of the control plasmid pSH995 or the fucosylation pathway plasmid pSH1022 is digested with the restriction enzyme SfiI to linearize the vector and transformed by electroporation into the host strain RDP974. The transformed cells are plated on YPD containing 100 ng/mL nourseothricin and incubated at 26° C. for five days. Subsequently several clones are picked and analyzed for fucose transfer onto the N-glycans of rEPO. A strain transformed with the control vector is designated YSH660, while a strain transformed with pSH1022 and demonstrating fucose transfer is designated YSH661.

Typically, protein expression is carried out by growing the transformed strains at 26° C. in 50 mL buffered glycerol-complex medium (BMGY) consisting of 1% yeast extract, 2% peptone, 100 mM potassium phosphate buffer, pH 6.0, 1.34% yeast nitrogen base, 4×10−5% biotin, and 1% glycerol as a growth medium. Induction of protein expression is performed in 5 mL of buffered methanol-complex medium (BMMY), consisting of 1.5% methanol instead of glycerol in BMGY.

Recombinant rEPO is expressed as described above and Ni-chelate column purified as described in Choi et al. (Proc. Natl. Acad. Sci. USA 100, 5022 (2003) and Hamilton et al. (Science 301, 1244 (2003)). The resultant protein is analyzed by SDS-PAGE (Laemmli, Nature 227, 680 (1970)) and stained for visualization with coomassie blue. Fucose is removed by in vitro digestion with α-1,6-fucosidase (Sigma-Aldrich, St. Louis, Mo.) treatment, as recommended by the manufacturer.

For glycan analysis, the glycans are released from rEPO by treatment with PNGase-F (Choi et al. (2003); Hamilton et al. (2003)). Released glycans are analyzed by MALDI/Time-of-flight (TOF) mass spectrometry to confirm glycan structures (Choi et al. (2003)). To quantitate the relative amount of fucosylated glycans present, the N-glycosidase F released glycans are labeled with 2-aminobenzidine (2-AB) and analyzed by HPLC (Choi et al. (2003)). The percentage of fucosylated and non-fucosylated glycans is calculated by comparing the peak area of each species before and after fucosidase treatment.

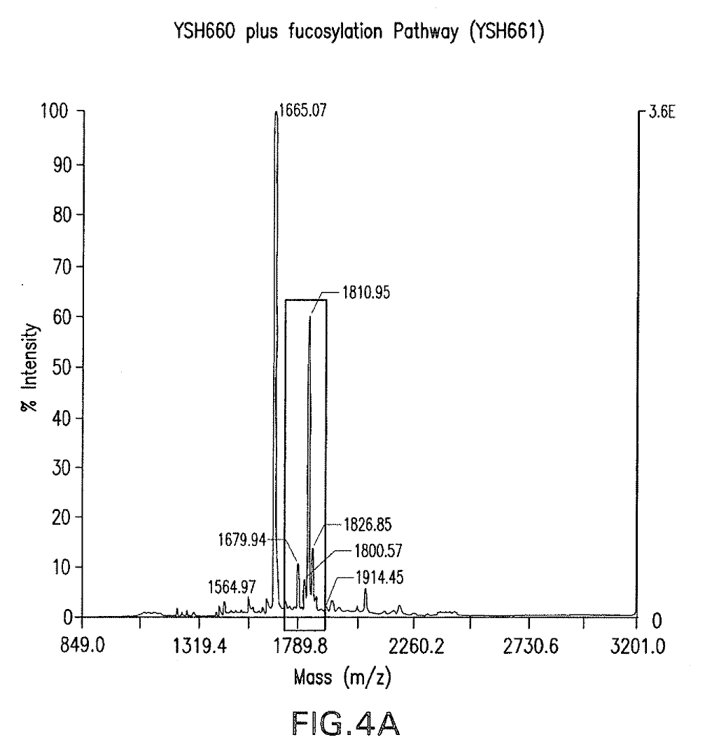

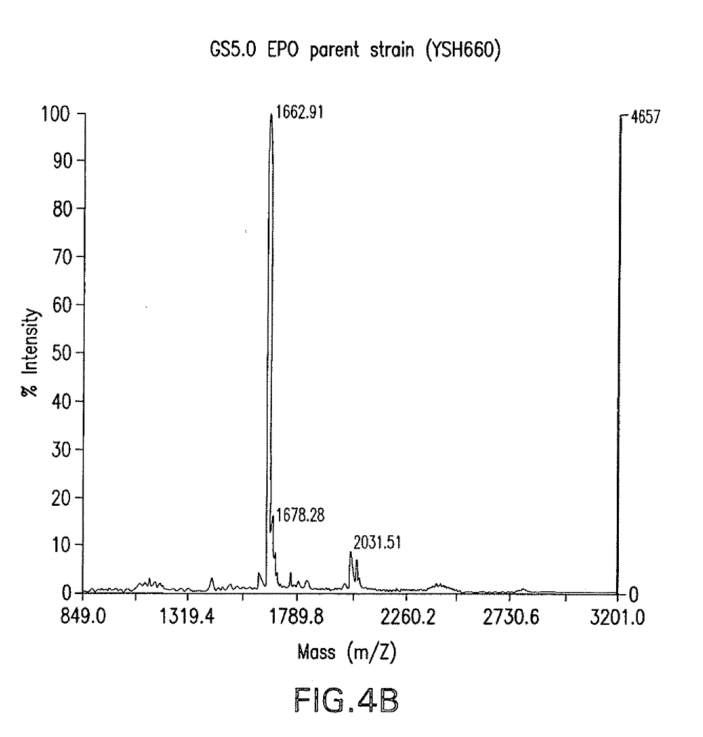

Analysis of the N-glycans produced in strain YSH661 produced essentially as described above showed that the strain produced recombinant rEPO comprising Gal₂GlcNAc₂Man₃GlcNAc₂(Fuc) N-glycans. FIG. 4A, which shows the results of a MALDI-TOF analysis of the N-glycans on rEPO produced in strain YSH661, shows that the strain produced N-glycans comprised Gal₂GlcNAc₂Man₃GlcNAc₂(Fuc) Gal₂GlcNAc₂Man₃GlcNAc₂ N-glycans. The Gal₂GlcNAc₂Man₃GlcNAc₂(Fuc) N-glycans are within the box. FIG. 4B, which shows a MALDI-TOF analysis of the N-glycans on rEPO produced in control strain YSH660 (without fucosylation pathway), shows that the strain produced only a fucosylated N-glycans comprising only Gal₂GlcNAc₂Man₃GlcNAc₂.

### Example 2

A Pichia pastoris strain capable of producing glycoproteins having NANA₂Gal₂GlcNAc₂Man₃GlcNAc₂(Fuc) N-glycans can be made by introducing the vector pSH1022 into a Pichia pastoris strain capable of producing glycoproteins having NANA₂Gal₂GlcNAc₂Man₃GlcNAc₂ N-glycans. For example, vector pSH1022 containing the genes encoding the components of the fucosylation pathway can be transformed into the strain YSH597, which produces rat EPO having NANA₂Gal₂GlcNAc₂Man₃GlcNAc₂ N-glycans and is disclosed in U.S. Provisional Application No. 60/801,688 and Hamilton et al. Science 313, 1441-1443 (2006). The rat EPO produced in the strain upon induction will include NANA₂Gal₂GlcNAc₂Man₃GlcNAc₂(Fuc) N-glycans.

The following provides a prophetic method for introducing the enzymes encoding the sialylation pathway into strain YSH661 of Example 1.

Open reading frames for Homo sapiens UDP-N-acetylglucosamine-2-epimerase/N-acetylmannosamine kinase (GNE), H. sapiens N-acetylneuraminate-9-phosphate synthase (SPS), H. sapiens CMP-sialic acid synthase (CSS), Mus musculus CMP-sialic acid transporter (CST), and amino acids 40 to 403 of M. musculus α-2,6-sialyltransferase (ST) are analyzed by the GeneOptimizer software and codon-optimized for P. pastoris expression (GeneArt, Regensburg, Germany). The resultant synthetic DNAs for GNE, SPS, CSS and CST are generated with 5′ BsaI and 3′ HpaI restriction sites, cloned into a shuttle vector and designated pGLY368, 367, 366 and 369, respectively. The synthetic DNA for ST is generated with 5′ AscI and 3′ PacI restriction sites, cloned into a shuttle vector, and designated pSH660. To generate the SPS, CSS and CST expression cassettes, the vectors pGLY367, 366 and 369 are digested with BsaI and HpaI to excise 1.1, 1.3, and 1.0 Kb fragments, which are treated with T4 DNA polymerase to remove single strandoverhangs. Subsequently, these fragments are subcloned into the vectors pGLY359, 17, and 363 previously digested with NotI and AscI for the former, and NotI and PacI for the latter two, and treated with T4 DNA polymerase. The resultant vectors pSH819, containing SPS in a PpPMA1prom-PpPMA1tt cassette flanked by PacI restriction sites; pSH824, containing CSS in a PpGAPDH-ScCYCtt cassette flanked by 5′ BglII and 3′ BamHI restriction sites; and pGLY372, containing CST in a PpPMA1prom-PpPMA1tt cassette flanked by RsrII restriction sites. To generate the ST catalytic domain fused to a yeast localization signal, the S. cerevisiae targeting region of Mnt1 is amplified from genomic DNA using Taq DNA polymerase (Promega, Madison, Wis.) and the primers ScMnt1- for and ScMnt1-rev (5′-GGGCGGCCGCCACCATGGCCCTCTTTCTC AGTAAGAGACT GTTGAG-3′ (SEQ ID NO:25) and 5′-CCGGCGCGCCCGATGACTTGTTG TTCAGGGGATATAGATCCTG-3′ (SEQ ID NO:26), respectively). The conditions used are: 94° C. for 3 minutes, 1 cycle; 94° C. for 30 seconds, 55° C. for 20 seconds, 68° C. for 1 minute, 30 cycles; 68° C. for 5 minutes, 1 cycle. The resultant 174 bp fragment containing 5′ NotI and 3′ AscI restriction sites is subcloned in-frame 5′ to the codon-optimized ST, creating the vector pSH861. Subsequently this vector is digested with NotI and PacI to excise a 1.3 Kb fragment, containing the ST-fusion, treated with T4 DNA polymerase and subcloned into pGLY361 prepared by digestion with NotI and PacI, and treated with T4 DNA polymerase. The resultant vector, containing the ST-fusion in a PpTEFprom-PpTEFtt cassette flanked by SgfI restriction sites, is designated pSH893.

A yeast expression vector pSH823, containing a P. pastoris GAPDH promoter and S. cerevisiae CYC transcription terminator, is designed to integrate into the Pichia genome downstream of the Trp2 ORF. The 2.6 Kb fragment encoding the PMA-CST expression cassette is excised from pGLY372 using the restriction enzyme RsrII and subcloned into pSH823 digested with the same enzyme. The resultant vector in which the PMA-CST and GAPDH expression cassettes are aligned in the same direction was designated pSH826. Subsequently this vector is digested with the restriction enzymes NotI and PacI and the single strand overhangs removed with T4 DNA polymerase. Into this linearized construct, the 2.2 Kb fragment of GNE, isolated from pGLY368 by digestion with BsaI and HpaI, and treated with T4 DNA polymerase to remove single strand overhangs, is subcloned. This vector is designated pSH828. Subsequently this vector is digested with PacI, into which the 2.7 Kb PacI fragment of pSH819, encoding the PMA-SPS expression cassette, is subcloned. The vector produced, in which the PMA-SPS expression cassette is aligned in the opposite orientation to the GAPDH expression cassette, is designated pSH830. At this stage the URA5 marker is replaced with HIS1 by excising the 2.4 Kb URA5 fragment from pSH830 using XhoI and replacing it with the 1.8 Kb fragment of HIS1 from pSH842 digested with the same enzyme. The resultant vector in which the HIS1 ORF is aligned in the same direction as GAPDH-GNE expression cassette is designated pSH870. Subsequently, this vector is digested with BamHI and the 2.1 Kb fragment from pSH824 isolated by digestion with BamHI and BglII, containing the GAPDH-CSS expression cassette, is subcloned. The vector generated, in which the newly introduced expression cassette is orientated in the opposite direction as the GAPDH-GNE cassette, is designated pSH872. Next, the 2.2 Kb expression cassette containing the TEF-ST is digested with SgfI from pSH893 and subcloned into pSH872 digested with the same enzyme. The vector generated, in which the TEF-ST cassette is orientated in the opposite direction as the GAPDH-GNE cassette, is designated pSH926.

The pSH926 vector is transformed into strain YSH661, which is then capable of producing rat EPO having NANA₂Gal₂GlcNAc₂Man₃GlcNAc₂ N-glycans.

### Example 3

A Pichia pastoris strain capable of producing a human EPO having NANA₂Gal₂GlcNAc₂Man₃GlcNAc₂(Fuc) N-glycans can be made by introducing the vector pSH1022 into a Pichia pastoris strain capable of producing human EPO having NANA₂Gal₂GlcNAc₂Man₃GlcNAc₂ N-glycans. For example, vector pSH1022 containing the genes encoding the components of the fucosylation pathway can be transformed into a strain that is capable of producing glycoproteins having NANA₂Gal₂GlcNAc₂Man₃GlcNAc₂ N-glycans, such as strain YSH597 disclosed in Hamilton et al., Science 313, 1441-1443 (2006) or YSH661 of Example 2 comprising the genes encoding the sialylation pathway enzymes but replacing the DNA encoding rat EPO with DNA encoding the human EPO. The strain will then produce human EPO having NANA₂Gal₂GlcNAc₂Man₃GlcNAc₂(Fuc) N-glycans.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A recombinant lower eukaryote host cell comprising a fucosylation pathway.

2. The host cell of claim 1 which is yeast or filamentous fungus.

3. The host cell of claim 2 wherein the yeast is a Pichia sp.

4. The host cell of claim 3 wherein the Pichia sp. is Pichia pastoris.

5. The host cell of claim 1 wherein the host cell further does not display α1,6-mannosyltransferase activity with respect to the N-glycan on a glycoprotein and includes an α1,2-mannosidase catalytic domain fused to a cellular targeting signal peptide not normally associated with the catalytic domain and selected to target α1,2-mannosidase activity to the ER or Golgi apparatus of the host cell whereby, upon passage of a recombinant glycoprotein through the ER or Golgi apparatus of the host cell, a recombinant glycoprotein comprising a fucosylated Man₅GlcNAc₂ glycoform is produced.

6. The host cell of claim 5 further including a GlcNAc transferase I catalytic domain fused to a cellular targeting signal peptide not normally associated with the catalytic domain of and selected to target GlcNAc transferase I activity to the ER or Golgi apparatus of the host cell; whereby, upon passage of the recombinant glycoprotein through the ER or Golgi apparatus of the host cell, a recombinant glycoprotein comprising a fucosylated GlcNAcMan₅GlcNAc₂ glycoform is produced.

7. The host cell of claim 6 further including a mannosidase II catalytic domain fused to a cellular targeting signal peptide not normally associated with the catalytic domain and selected to target mannosidase II activity to the ER or Golgi apparatus of the host cell; whereby, upon passage of the recombinant glycoprotein through the ER or Golgi apparatus of the host cell, a recombinant glycoprotein comprising a fucosylated GlcNAcMan₃GlcNAc₂ glycoform is produced.

8. The host cell of claim 7 further including a GlcNAc transferase II catalytic domain fused to a cellular targeting signal peptide not normally associated with the catalytic domain and selected to target GlcNAc transferase II activity to the ER or Golgi apparatus of the host cell; whereby, upon passage of the recombinant glycoprotein through the ER or Golgi apparatus of the host cell, a recombinant glycoprotein comprising a fucosylated GlcNAc₂Man₃GlcNAc₂ glycoform is produced.

9. The host cell of claim 8 further including a galactosyltransferase catalytic domain fused to a cellular targeting signal peptide not normally associated with the catalytic domain and selected to target Galactose transferase II activity to the ER or Golgi apparatus of the host cell; whereby, upon passage of the recombinant glycoprotein through the ER or Golgi apparatus of the host cell, a recombinant glycoprotein comprising a fucosylated GalGlcNAc₂Man₃GlcNAc₂ or Gal₂GlcNAc₂Man₃GlcNAc₂ glycoform is produced.

10. The host cell of claim 9 further including a sialyltransferase catalytic domain fused to a cellular targeting signal peptide not normally associated with the catalytic domain and selected to target sialyltransferase activity to the ER or Golgi apparatus of the host cell; whereby, upon passage of the recombinant glycoprotein through the ER or Golgi apparatus of the host cell, a recombinant glycoprotein comprising a fucosylated NANAGal₂GlcNAc₂Man₃GlcNAc₂ or NANA₂Gal₂GlcNAc₂Man₃GlcNAc₂ glycoform is produced.

11-19. (canceled)

20. A hybrid vector comprising (a) DNA regulatory elements which are functional in a lower eukaryotic host cell operatively linked to (b) DNA coding sequence encoding a fusion protein encoding (i) a targeting sequence; and (b) a catalytic domain of a fucosylation pathway enzyme.

21. The vector of claim 20 wherein the fucosylation pathway enzyme is a fucosyltransferase.

22. The host cell of claim 1, wherein the fucosylation pathway comprises a GDP-mannose-4,6-dehydratase, GDP-keto-deoxy-mannose-epimerase/GDP-keto-deoxy-galactose-reductase, GDP-fucose transporter, and a fucosyltransferase.

23. The host cell of claim 22, wherein the fucosyltransferase is selected from the group consisting of α1,2-fucosyltransferase, α1,3-fucosyltransferase, α1,4-fucosyltransferase, and α1,6-fucosyltransferase.

24. A method of producing a glycoprotein in a lower eukaryote comprising one or more fucosylated N-glycans comprising:
(a) providing a lower eukaryote host cell comprising a fucosylation pathway and capable of producing hybrid or complex N-glycans and which has been transformed with a nucleic acid molecule encoding the glycoprotein; and
(b) cultivating the host cell under conditions for expression of the heterologous glycoprotein to produce the glycoprotein comprising one or more fucosylated N-glycans.

25. The method of claim 24, wherein the fucosylation pathway comprises a GDP-mannose-4,6-dehydratase, GDP-keto-deoxy-mannose-epimerase/GDP-keto-deoxy-galactose-reductase, GDP-fucose transporter, and a fucosyltransferase.

26. The host cell of claim 25, wherein the fucosyltransferase is selected from the group consisting of α1,2-fucosyltransferase, α1,3-fucosyltransferase, α1,4-fucosyltransferase, and α1,6-fucosyltransferase.

27. The method of claim 24, wherein the glycoprotein is a therapeutic glycoprotein.

28. The method of claim 24, wherein the glycoprotein is selected from the group consisting of erythropoietin (EPO); cytokines such as interferon-α, interferon-β, interferon-γ, interferon-ω, and granulocyte-CSF; coagulation factors such as factor VIII, factor IX, and human protein C; monoclonal antibodies, soluble IgE receptor α-chain, IgG, IgM, IgG, urokinase, chymase, and urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, osteoprotegerin tissue, plasminogen activator, G-CSF, GM-CSF, and TNF-receptor.

29. The method of claim 24, wherein the host cell is a yeast or filamentous fungus.

30. The method of claim 24, wherein the host cell is a Pichia sp.

31. The method of claim 24, wherein the host cell is Pichia pastoris.

32. A glycoprotein composition comprising one or more glycoproteins produced by the method of claim 24.

33. The host cell of claim 7, wherein the host cell further includes one or more GlcNAc transferases selected from the group consisting of GnTIII, GnTIV, GnTV, GnTVI, and GnTIX.

34. The host cell of claim 8, wherein the host cell further includes one or more GlcNAc transferases selected from the group consisting of GnTIII, GnTIV, GnTV, GnTVI, and GnTIX.

35. The host cell of claim 9, wherein the host cell further includes one or more GlcNAc transferases selected from the group consisting of GnTIII, GnTIV, GnTV, GnTVI, and GnTIX.

36. The host cell of claim 10, wherein the host cell further includes one or more GlcNAc transferases selected from the group consisting of GnTIII, GnTIV, GnTV, GnTVI, and GnTIX.


SEQUENCE LISTING (26 sequences)

SEQ ID NO: 1 - DNA, 1119 bp, Homo sapians
  CDS (1)...(1119): GDP-Mannose-dehydratase (hGMD)
atggcacacgcaccggcacgctgccccagcgcccggggctccggggacggcgagatgggcaagcccaggaacgtggcgctcatcaccggtatcacaggccaggatggttcctacctggctgagttcctgctggagaaaggctatgaggtccatggaattgtacggcggtccagttcatttaatacgggtcgaattgagcatctgtataagaatccccaggctcacattgaaggaaacatgaagttgcactatggcgatctcactgacagtacctgccttgtgaagatcattaatgaagtaaagcccacagagatctacaaccttggagcccagagccacgtcaaaatttcctttgacctcgctgagtacactgcggacgttgacggagttggcactctacgacttctagatgcagttaagacttgtggccttatcaactctgtgaagttctaccaagcctcaacaagtgaactttatgggaaagtgcaggaaataccccagaaggagaccacccctttctatccccggtcaccctatggggcagcaaaactctatgcctattggattgtggtgaacttccgtgaggcgtataatctctttgcagtgaacggcattctcttcaatcatgagagtcccagaagaggagctaatttcgttactcgaaaaattagccggtcagtagctaagatttaccttggacaactggaatgtttcagtttgggaaatctggatgccaaacgagattggggccatgccaaggactatgtggaggctatgtggttgatgttgcagaatgatgagccggaggacttcgttatagctactggggaggtccatagtgtccgggaatttgtcgagaaatcattcttgcacattggaaaaaccattgtgtgggaaggaaagaatgaaaatgaagtgggcagatgtaaagagaccggcaaagttcacgtgactgtggatctcaagtactaccggccaactgaagtggactttctgcagggcgactgcaccaaagcgaaacagaagctgaactggaagccccgggtcgctttcgatgagctggtgagggagatggtgcacgccgacgtggagctcatgaggacaaaccccaatgcctga

SEQ ID NO: 2 - protein, 372 aa, Homo sapians
MAHAPARCPSARGSGDGEMGKPRNVALITGITGQDGSYLAEFLLEKGYEVHGIVRRSSSFNTGRIEHLYKNPQAHIEGNMKLHYGDLTDSTCLVKIINEVKPTEIYNLGAQSHVKISFDLAEYTADVDGVGTLRLLDAVKTCGLINSVKFYQASTSELYGKVQEIPQKETTPFYPRSPYGAAKLYAYWIVVNFREAYNLFAVNGILFNHESPRRGANFVTRKISRSVAKIYLGQLECFSLGNLDAKRDWGHAKDYVEAMWLMLQNDEPEDFVIATGEVHSVREFVEKSFLHIGKTIVWEGKNENEVGRCKETGKVHVTVDLKYYRPTEVDFLQGDCTKAKQKLNWKPRVAFDELVREMVHADVELMRTNPNA

SEQ ID NO: 3 - DNA, 966 bp, Homo sapians
  CDS (1)...(966): GDP-ketoxy-deoxy-mannose-epimerase/GDP- keto-deoxy-galactose-reductase (FX protein)
atgggtgaaccccagggatccatgcggattctagtgacagggggctctgggctggtaggcaaagccatccagaaggtggtagcagatggagctggacttcctggagaggactgggtgtttgtctcctctaaagacgccgatctcacggatacagcacagacccgcgccctgtttgagaaggtccaacccacacacgtcatccatcttgctgcaatggtggggggcctgttccggaatatcaaatacaatttggacttctggaggaaaaacgtgcacatgaacgacaacgtcctgcactcggcctttgaggtgggggcccgcaaggtggtgtcctgcctgtccacctgtatcttccctgacaagacgacctacccgatagatgagaccatgatccacaatgggcctccccacaacagcaattttgggtactcgtatgccaagaggatgatcgacgtgcagaacagggcctacttccagcagtacggctgcaccttcaccgctgtcatccccaccaacgttttcgggccccacgacaacttcaacatcgaggatggccacgtgctgcctggcctcatccacaaggtgcacctggccaagagcagcggctcggccctgacggtgtggggtacagggaatccgcggaggcagttcatatactcgctggacctggcccagctctttatctgggtcctgcgggagtacaatgaagtggagcccatcatcctctccgtgggcgaggaagatgaggtctccatcaaggaggcagccgaggcggtggtggaggccatggacttccatggggaagtcacctttgatacaaccaagtcggatgggcagtttaagaagacagccagtaacagcaagctgaggacctacctgcccgacttccggttcacacccttcaagcaggcggtgaaggagacctgtgcttggttcactgacaactacgagcaggcccggaagtga

SEQ ID NO: 4 - protein, 321 aa, Homo sapians
MGEPQGSMRILVTGGSGLVGKAIQKVVADGAGLPGEDWVFVSSKDADLTDTAQTRALFEKVQPTHVIHLAAMVGGLFRNIKYNLDFWRKNVHMNDNVLHSAFEVGARKVVSCLSTCIFPDKTTYPIDETMIHNGPPHNSNFGYSYAKRMIDVQNRAYFQQYGCTFTAVIPTNVFGPHDNFNIEDGHVLPGLIHKVHLAKSSGSALTVWGTGNPRRQFIYSLDLAQLFIWVLREYNEVEPIILSVGEEDEVSIKEAAEAVVEAMDFHGEVTFDTTKSDGQFKKTASNSKLRTYLPDFRFTPFKQAVKETCAWFTDNYEQARK

SEQ ID NO: 5 - DNA, 1095 bp, Homo sapians
  CDS (1)...(1095): GDP-fucose transporter
atgaatagggcccctctgaagcggtccaggatcctgcacatggcgctgaccggggcctcagacccctctgcagaggcagaggccaacggggagaagccctttctgctgcgggcattgcagatcgcgctggtggtctccctctactgggtcacctccatctccatggtgttccttaataagtacctgctggacagcccctccctgcggctggacacccccatcttcgtcaccttctaccagtgcctggtgaccacgctgctgtgcaaaggcctcagcgctctggccgcctgctgccctggtgccgtggacttccccagcttgcgcctggacctcagggtggcccgcagcgtcctgcccctgtcggtggtcttcatcggcatgatcaccttcaataacctctgcctcaagtacgtcggtgtggccttctacaatgtgggccgctcactcaccaccgtcttcaacgtgctgctctcctacctgctgctcaagcagaccacctccttctatgccctgctcacctgcggtatcatcatcgggggcttctggcttggtgtggaccaggagggggcagaaggcaccctgtcgtggctgggcaccgtcttcggcgtgctggctagcctctgtgtctcgctcaacgccatctacaccacgaaggtgctcccggcggtggacggcagcatctggcgcctgactttctacaacaacgtcaacgcctgcatcctcttcctgcccctgctcctgctgctcggggagcttcaggccctgcgtgactttgcccagctgggcagtgcccacttctgggggatgatgacgctgggcggcctgtttggctttgccatcggctacgtgacaggactgcagatcaagttcaccagtccgctgacccacaatgtgtcgggcacggccaaggcctgtgcccagacagtgctggccgtgctctactacgaggagaccaagagcttcctctggtggacgagcaacatgatggtgctgggcggctcctccgcctacacctgggtcaggggctgggagatgaagaagactccggaggagcccagccccaaagacagcgagaagagcgccatgggggtgtga

SEQ ID NO: 6 - protein, 364 aa, Homo sapians
MNRAPLKRSRILHMALTGASDPSAEAEANGEKPFLLRALQIALVVSLYWVTSISMVFLNKYLLDSPSLRLDTPIFVTFYQCLVTTLLCKGLSALAACCPGAVDFPSLRLDLRVARSVLPLSVVFIGMITFNNLCLKYVGVAFYNVGRSLTTVFNVLLSYLLLKQTTSFYALLTCGIIIGGFWLGVDQEGAEGTLSWLGTVFGVLASLCVSLNAIYTTKVLPAVDGSIWRLTFYNNVNACILFLPLLLLLGELQALRDFAQLGSAHFWGMMTLGGLFGFAIGYVTGLQIKFTSPLTHNVSGTAKACAQTVLAVLYYEETKSFLWWTSNMMVLGGSSAYTWVRGWEMKKTPEEPSPKDSEKSAMGV

SEQ ID NO: 7 - DNA, 1728 bp, Homo sapians
  CDS (1)...(1728): alpha-1,6-fucosyltransferase (hFuT8)
atgcggccatggactggttcctggcgttggattatgctcattctttttgcctgggggaccttgctgttttatataggtggtcacttggtacgagataatgaccatcctgatcactctagccgagaactgtccaagattctggcaaagcttgaacgcttaaaacaacagaatgaagacttgaggcgaatggccgaatctctccggataccagaaggccctattgatcaggggccagctataggaagagtacgcgttttagaagagcagcttgttaaggccaaagaacagattgaaaattacaagaaacagaccagaaatggtctggggaaggatcatgaaatcctgaggaggaggattgaaaatggagctaaagagctctggtttttcctacagagtgaattgaagaaattaaagaacttagaaggaaatgaactccaaagacatgcagatgaatttcttttggatttaggacatcatgaaaggtctataatgacggatctatactacctcagtcagacagatggagcaggtgattggcgggaaaaagaggccaaagatctgacagaactggttcagcggagaataacatatcttcagaatcccaaggactgcagcaaagccaaaaagctggtgtgtaatatcaacaaaggctgtggctatggctgtcagctccatcatgtggtctactgcttcatgattgcatatggcacccagcgaacactcatcttggaatctcagaattggcgctatgctactggtggatgggagactgtatttaggcctgtaagtgagacatgcacagacagatctggcatctccactggacactggtcaggtgaagtgaaggacaaaaatgttcaagtggtcgagcttcccattgtagacagtcttcatccccgtcctccatatttacccttggctgtaccagaagacctcgcagatcgacttgtacgagtgcatggtgaccctgcagtgtggtgggtgtctcagtttgtcaaatacttgatccgcccacagccttggctagaaaaagaaatagaagaagccaccaagaagcttggcttcaaacatccagttattggagtccatgtcagacgcacagacaaagtgggaacagaagctgccttccatcccattgaagagtacatggtgcatgttgaagaacattttcagcttcttgcacgcagaatgcaagtggacaaaaaaagagtgtatttggccacagatgacccttctttattaaaggaggcaaaaacaaagtaccccaattatgaatttattagtgataactctatttcctggtcagctggactgcacaatcgatacacagaaaattcacttcgtggagtgatcctggatatacattttctctctcaggcagacttcctagtgtgtactttttcatcccaggtctgtcgagttgcttatgaaattatgcaaacactacatcctgatgcctctgcaaacttccattctttagatgacatctactattttgggggccagaatgcccacaatcaaattgccatttatgctcaccaaccccgaactgcagatgaaattcccatggaacctggagatatcattggtgtggctggaaatcattgggatggctattctaaaggtgtcaacaggaaattgggaaggacgggcctatatccctcctacaaagttcgagagaagatagaaacggtcaagtaccccacatatcctgaggctgagaaataa

SEQ ID NO: 8 - protein, 575 aa, Homo sapians
MRPWTGSWRWIMLILFAWGTLLFYIGGHLVRDNDHPDHSSRELSKILAKLERLKQQNEDLRRMAESLRIPEGPIDQGPAIGRVRVLEEQLVKAKEQIENYKKQTRNGLGKDHEILRRRIENGAKELWFFLQSELKKLKNLEGNELQRHADEFLLDLGHHERSIMTDLYYLSQTDGAGDWREKEAKDLTELVQRRITYLQNPKDCSKAKKLVCNINKGCGYGCQLHHVVYCFMIAYGTQRTLILESQNWRYATGGWETVFRPVSETCTDRSGISTGHWSGEVKDKNVQVVELPIVDSLHPRPPYLPLAVPEDLADRLVRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPSLLKEAKTKYPNYEFISDNSISWSAGLHNRYTENSLRGVILDIHFLSQADFLVCTFSSQVCRVAYEIMQTLHPDASANFHSLDDIYYFGGQNAHNQIAIYAHQPRTADEIPMEPGDIIGVAGNHWDGYSKGVNRKLGRTGLYPSYKVREKIETVKYPTYPEAEK

SEQ ID NO: 9 - DNA, 1728 bp, Rattus norvegicus
  CDS (1)...(1728): alpha-1,6-fucosyltransferase (rFuT8)
atgcgggcatggactggttcctggcgttggattatgctcattctttttgcctgggggaccttgttgttttatataggtggtcatttggttcgagataatgaccaccctgatcactctagcagagaactctccaagattcttgcaaagcttgaacgcttaaaacaacaaaatgaagacttgaggcgaatggctgagtctctacgaataccagaaggccccattgaccaggggacggctacgggaagagtccgtgttttagaagaacagcttgttaaggccaaagaacagattgaaaattacaagaaacaagccagaaatggtctggggaaggatcatgaactcttaaggaggaggattgaaaatggagctaaagagctctggttttttctacaaagtgaactgaagaaattaaagcatctagaaggaaatgaactccaaagacatgcagatgaaattcttttggatttaggacaccatgaaaggtctatcatgacggatctatactacctcagtcaaacagatggagcaggggattggcgtgaaaaagaggccaaagatctgacagagctggtccagcggagaataacttatctccagaatcccaaggactgcagcaaagccaggaagctggtgtgtaacatcaataagggctgtggctatggttgccaactccatcacgtggtctactgtttcatgattgcttatggcacccagcgaacactcatcttggaatctcagaattggcgctatgctactggtggatgggagactgtgtttagacctgtaagtgagacatgcacagacagatctggcctctccactggacactggtcaggtgaagtgaatgacaaaaatattcaagtggtggagctccccattgtagacagcctccatcctcggcctccttacttaccactggctgttccagaagaccttgcagatcgactcgtaagagtccatggtgatcctgcagtgtggtgggtgtcccagttcgtcaaatatttgattcgtccacaaccttggctagaaaaggaaatagaagaagccaccaagaagcttggcttcaaacatccagtcattggagtccatgtcagacgcacagacaaagtgggaacagaggcagccttccatcccatcgaagagtacatggtacatgttgaagaacattttcagcttctcgcacgcagaatgcaagtggataaaaaaagagtatatctggctaccgatgaccctgctttgttaaaggaggcaaagacaaagtactccaattatgaatttattagtgataactctatttcttggtcagctggattacacaatcggtacacagaaaattcacttcggggcgtgatcctggatatacactttctctctcaggctgacttcctagtgtgtactttttcatcccaggtctgtcgggttgcttatgaaatcatgcaaaccctgcatcctgatgcctctgcaaacttccactctttagatgacatctactattttggaggccaaaatgcccacaaccagattgccgtttatcctcacaaacctcgaactgatgaggaaattccaatggaacctggagatatcattggtgtggctggaaaccattgggatggttattctaaaggtgtcaacagaaaacttggaaaaacaggcttatatccctcctacaaagtccgagagaagatagaaacagtcaagtatcccacatatcctgaagctgaaaaatag

SEQ ID NO: 10 - protein, 575 aa, Rattus norvegicus
MRAWTGSWRWIMLILFAWGTLLFYIGGHLVRDNDHPDHSSRELSKILAKLERLKQQNEDLRRMAESLRIPEGPIDQGTATGRVRVLEEQLVKAKEQIENYKKQARNGLGKDHELLRRRIENGAKELWFFLQSELKKLKHLEGNELQRHADEILLDLGHHERSIMTDLYYLSQTDGAGDWREKEAKDLTELVQRRITYLQNPKDCSKARKLVCNINKGCGYGCQLHHVVYCFMIAYGTQRTLILESQNWRYATGGWETVFRPVSETCTDRSGLSTGHWSGEVNDKNIQVVELPIVDSLHPRPPYLPLAVPEDLADRLVRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPALLKEAKTKYSNYEFISDNSISWSAGLHNRYTENSLRGVILDIHFLSQADFLVCTFSSQVCRVAYEIMQTLHPDASANFHSLDDIYYFGGQNAHNQIAVYPHKPRTDEEIPMEPGDIIGVAGNHWDGYSKGVNRKLGKTGLYPSYKVREKIETVKYPTYPEAEK

SEQ ID NO: 11 - DNA, 1728 bp, Mus musculus
  CDS (1)...(1728): alpha-1,6-fucosyltransferase (mFuT8)
atgcgggcatggactggttcctggcgttggattatgctcattctttttgcctgggggaccttgttattttatataggtggtcatttggttcgagataatgaccaccctgatcactccagcagagaactctccaagattcttgcaaagcttgaacgcttaaaacagcaaaatgaagacttgaggcgaatggctgagtctctccgaataccagaaggccccattgaccaggggacagctacaggaagagtccgtgttttagaagaacagcttgttaaggccaaagaacagattgaaaattacaagaaacaagctagaaatggtctggggaaggatcatgaaatcttaagaaggaggattgaaaatggagctaaagagctctggttttttctacaaagcgaactgaagaaattaaagcatttagaaggaaatgaactccaaagacatgcagatgaaattcttttggatttaggacaccatgaaaggtctatcatgacagatctatactacctcagtcaaacagatggagcaggggattggcgtgaaaaagaggccaaagatctgacagagctggtccagcggagaataacatatctccagaatcctaaggactgcagcaaagccaggaagctggtgtgtaacatcaataaaggctgtggctatggttgtcaactccatcacgtggtctactgtttcatgattgcttatggcacccagcgaacactcatcttggaatctcagaattggcgctatgctactggtggatgggagactgtgtttagacctgtaagtgagacatgtacagacagatctggcctctccactggacactggtcaggtgaagtaaatgacaaaaacattcaagtggtcgagctccccattgtagacagcctccatcctcggcctccttacttaccactggctgttccagaagaccttgcagaccgactcctaagagtccatggtgaccctgcagtgtggtgggtgtcccagtttgtcaaatacttgattcgtccacaaccttggctggaaaaggaaatagaagaagccaccaagaagcttggcttcaaacatccagttattggagtccatgtcagacgcacagacaaagtgggaacagaagcagccttccaccccatcgaggagtacatggtacacgttgaagaacattttcagcttctcgcacgcagaatgcaagtggataaaaaaagagtatatctggctactgatgatcctactttgttaaaggaggcaaagacaaagtactccaattatgaatttattagtgataactctatttcttggtcagctggactacacaatcggtacacagaaaattcacttcggggtgtgatcctggatatacactttctctcacaggctgactttctagtgtgtactttttcatcccaggtctgtcgggttgcttatgaaatcatgcaaaccctgcatcctgatgcctctgcgaacttccattctttggatgacatctactattttggaggccaaaatgcccacaatcagattgctgtttatcctcacaaacctcgaactgaagaggaaattccaatggaacctggagatatcattggtgtggctggaaaccattgggatggttattctaaaggtatcaacagaaaacttggaaaaacaggcttatatccctcctacaaagtccgagagaagatagaaacagtcaagtatcccacatatcctgaagctgaaaaatag

SEQ ID NO: 12 - protein, 575 aa, Mus musculus
MRAWTGSWRWIMLILFAWGTLLFYIGGHLVRDNDHPDHSSRELSKILAKLERLKQQNEDLRRMAESLRIPEGPIDQGTATGRVRVLEEQLVKAKEQIENYKKQARNGLGKDHEILRRRIENGAKELWFFLQSELKKLKHLEGNELQRHADEILLDLGHHERSIMTDLYYLSQTDGAGDWREKEAKDLTELVQRRITYLQNPKDCSKARKLVCNINKGCGYGCQLHHVVYCFMIAYGTQRTLILESQNWRYATGGWETVFRPVSETCTDRSGLSTGHWSGEVNDKNIQVVELPIVDSLHPRPPYLPLAVPEDLADRLLRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPTLLKEAKTKYSNYEFISDNSISWSAGLHNRYTENSLRGVILDIHFLSQADFLVCTFSSQVCRVAYEIMQTLHPDASANFHSLDDIYYFGGQNAHNQIAVYPHKPRTEEEIPMEPGDIIGVAGNHWDGYSKGINRKLGKTGLYPSYKVREKIETVKYPTYPEAEK

SEQ ID NO: 13 - DNA, 1728 bp, Sus scrofa
  CDS (1)...(1728): alpha-1,6-fucosyltransferase (pFuT8)
atgcggccatggactggttcgtggcgttggattatgctcattctttttgcctgggggaccttgctattttacataggtggtcacttggtacgagataatgaccactctgatcactctagccgagaactgtccaagattttggcaaagctggaacgcttaaaacaacaaaatgaagacttgaggagaatggctgaatctctccgaataccagaaggccccattgatcaggggccagcttcaggaagagttcgtgctttagaagagcaatttatgaaggccaaagaacagattgaaaattataagaaacaaactaaaaatggtccagggaaggatcatgaaatcctaaggaggaggattgaaaatggagctaaagagctctggttttttctacaaagtgagttgaagaaattaaagaatttagaaggaaatgaactccaaagacatgcagatgaatttctatcagatttgggacatcatgaaaggtctataatgacggatctatactacctcagtcaaacagatggggcaggtgattggcgtgaaaaggaggccaaagatctgacagagctggtccagcggagaataacatatcttcagaatcccaaggactgcagcaaagccaagaagctagtgtgtaatatcaacaaaggctgtggctatggctgtcagctccatcatgtagtgtactgctttatgattgcatatggcacccagcgaacactcgccttggaatctcacaattggcgctacgctactgggggatgggaaactgtgtttagacctgtaagtgagacgtgcacagacagatctggcagctccactggacattggtcaggtgaagtaaaggacaaaaatgttcaggtggttgagctccccattgtagacagtgttcatcctcgtcctccatatttacccctggctgtcccagaagaccttgcagatcgacttgtacgagtccatggtgatcctgcagtgtggtgggtatcccagtttgtcaagtacttgattcgcccacaaccctggctggaaaaggaaatagaagaggccaccaagaagctaggcttcaaacatccagttattggagtccatgttagacgcacagacaaagtgggagcggaagcagccttccatcccattgaggaatacacggtgcacgttgaagaagactttcagcttcttgctcgcagaatgcaagtggataaaaaaagggtgtatttggccacagatgaccctgctttgttaaaagaggcaaaaacaaagtaccccagttatgaatttattagtgataactctatctcttggtcagctggactacataatcgatatacagaaaattcacttcggggtgtgatcctggatatacactttctctcccaggcagacttcctagtgtgtactttttcatcgcaggtctgtagagttgcttatgaaatcatgcaagcgctgcatcctgatgcctctgcgaacttccgttctttggatgacatctactattttggaggcccaaatgcccacaaccaaattgccatttatcctcaccaacctcgaactgaaggagaaatccccatggaacctggagatattattggtgtggctggaaatcactgggatggctatcctaaaggtgttaacagaaaactgggaaggacgggcctatatccctcctacaaagttcgagagaagatagaaacagtcaagtaccccacatatcccgaggctgacaagtaa

SEQ ID NO: 14 - protein, 575 aa, Sus scrofa
MRPWTGSWRWIMLILFAWGTLLFYIGGHLVRDNDHSDHSSRELSKILAKLERLKQQNEDLRRMAESLRIPEGPIDQGPASGRVRALEEQFMKAKEQIENYKKQTKNGPGKDHEILRRRIENGAKELWFFLQSELKKLKNLEGNELQRHADEFLSDLGHHERSIMTDLYYLSQTDGAGDWREKEAKDLTELVQRRITYLQNPKDCSKAKKLVCNINKGCGYGCQLHHVVYCFMIAYGTQRTLALESHNWRYATGGWETVFRPVSETCTDRSGSSTGHWSGEVKDKNVQVVELPIVDSVHPRPPYLPLAVPEDLADRLVRVHGDPAVWWVSQFVKYLIRPQPWLEKEIEEATKKLGFKHPVIGVHVRRTDKVGAEAAFHPIEEYTVHVEEDFQLLARRMQVDKKRVYLATDDPALLKEAKTKYPSYEFISDNSISWSAGLHNRYTENSLRGVILDIHFLSQADFLVCTFSSQVCRVAYEIMQALHPDASANFRSLDDIYYFGGPNAHNQIAIYPHQPRTEGEIPMEPGDIIGVAGNHWDGYPKGVNRKLGRTGLYPSYKVREKIETVKYPTYPEADK

SEQ ID NO: 15 - DNA, 37 bp, Artificial Sequence
  PCR primer SH415
ggcggccgccaccatggcacacgcaccggcacgctgc

SEQ ID NO: 16 - DNA, 33 bp, Artificial Sequence
  PCR primer SH413
ttaattaatcaggcattggggtttgtcctcatg

SEQ ID NO: 17 - DNA, 37 bp, Artificial Sequence
  PCR primer SH414
ggcggccgccaccatgggtgaaccccagggatccatg

SEQ ID NO: 18 - DNA, 33 bp, Artificial Sequence
  PCR primer SH411
ttaattaatcacttccgggcctgctcgtagttg

SEQ ID NO: 19 - DNA, 36 bp, Artificial Sequence
  PCR primer RCD679
gcggccgccaccatgaatagggcccctctgaagcgg

SEQ ID NO: 20 - DNA, 32 bp, Artificial Sequence
  PCR primer RCD680
ttaattaatcacacccccatggcgctcttctc

SEQ ID NO: 21 - DNA, 36 bp, Artificial Sequence
  PCR primer SH420
gcggcgcgccgataatgaccaccctgatcactccag

SEQ ID NO: 22 - DNA, 37 bp, Artificial Sequence
  PCR primer SH421
ccttaattaactatttttcagcttcaggatatgtggg

SEQ ID NO: 23 - DNA, 32 bp, Artificial Sequence
  PCR primer rEPO-forward
gggaattcgctcccccacgcctcatttgcgac

SEQ ID NO: 24 - DNA, 32 bp, Artificial Sequence
  PCR primer rEPO-reverse
cctctagatcacctgtcccctctcctgcaggc

SEQ ID NO: 25 - DNA, 46 bp, Artificial Sequence
  PCR primer ScMntI-forward
gggcggccgccaccatggccctctttctcagtaagagactgttgag

SEQ ID NO: 26 - DNA, 43 bp, Artificial Sequence
  PCR primer ScMnt1-reverse
ccggcgcgcccgatgacttgttgttcaggggatatagatcctg